(19) United States
(12) Patent Application Publication
(10) Pub. No.: US 2026/0315062 A1
(43) Pub. Date: Oct. 15, 2026

(54) APPARATUS AND METHOD FOR ADJUSTABLY SECURING A SUTURE TO A BONE ANCHOR

(71) Applicant: Simparo Inc., Mobile, AL (US)

(72) Inventors: Sidney Fleischman, Durham, NC (US); James Whayne, Chapel Hill, NC (US); Izi Bruker, Newton, MA (US); Jeanne L. DelSignore, Rushville, NY (US); Kevin L. Ohashi, Jamaica Plain, MA (US); James Christopher Harber, Shorewood, MN (US); Richard Briganti, Mobile, AL (US)

(21) Appl. No.: 19/717,210

(22) Filed: Jun. 23, 2026

Related U.S. Application Data
(63) Continuation-in-part of application No. 17/978,589, filed on Nov. 1, 2022, now Pat. No. 12,661,103.
Application No. 17/978,589 is a continuation of application No. 15/999,085, filed on Aug. 16, 2018, now Pat. No. 11,484,301.
(60) Provisional application No. 62/546,187, filed on Aug. 16, 2017.

(51) Int. Cl. A61B 17/04; A61B 17/56; A61B 17/86; A61F 2/08; A61F 2/30; A61F 2/42

(52) U.S. Cl. CPC A61B 17/0401; A61B 17/0487; A61B 17/8695; A61F 2/0811; A61F 2/4202; A61F 2/4241; A61B 2017/0409; A61B 2017/0414; A61B 2017/044; A61B 2017/0448; A61B 2017/0456; A61B 2017/0459; A61B 2017/0462; A61B 2017/565; A61B 2017/567; A61F 2002/0829; A61F 2002/0864; A61F 2002/0882; A61F 2002/0888; A61F 2002/30461; A61F 2002/30672; A61F 2002/3085; A61F 2002/30998; A61F 2002/4253

(57) ABSTRACT

Apparatus for adjustably securing an object to a washer body, the apparatus comprising: a washer body having a central opening; a first passageway extending through the washer body on a first side of the central opening and a second passageway extending through the washer body on a second, opposite side of the central opening; and a first suture-securing structure disposed in the first passageway and a second suture-securing structure disposed in the second passageway, wherein each of the first suture-securing structure and the second suture-securing structure comprise a side wall defining a suture passageway, a portion of the side wall projecting into the passageway so as to provide one-way movement of a suture through the suture passageway.

### REFERENCE TO PENDING PRIOR PATENT APPLICATIONS

This patent application:
(i) is a continuation-in-part of pending prior U.S. patent application Ser. No. 17/978,589, filed Nov. 1, 2022 by Simparo Inc. and Sidney Fleischman et al. for SUTURE-LOCKING WASHER FOR USE WITH A BONE ANCHOR, AND METHOD FOR SUPPORTING THE THUMB OF A PATIENT AFTER BASAL JOINT ARTHROPLASTY, AND OTHER NOVEL ORTHOPEDIC APPARATUS AND OTHER NOVEL ORTHOPEDIC PROCEDURES (Attorney's Docket No. FENTON-2225 CON), which patent application in turn:
   (1) is a continuation of prior U.S. patent application Ser. No. 15/999,085, filed Aug. 16, 2018 by CMC Group LLC and Sidney Fleischman et al. for SUTURE-LOCKING WASHER FOR USE WITH A BONE ANCHOR, AND METHOD FOR SUPPORTING THE THUMB OF A PATIENT AFTER BASAL JOINT ARTHROPLASTY, AND OTHER NOVEL ORTHOPEDIC APPARATUS AND OTHER NOVEL ORTHOPEDIC PROCEDURES (Attorney's Docket No. FENTON-2225), which patent application in turn:
      (a) claims benefit of prior U.S. Provisional Patent Application Ser. No. 62/546,187, filed Aug. 16, 2017 by CMC Group LLC and Sidney Fleischman et al. for SUTURE-LOCKING WASHER FOR USE WITH A BONE ANCHOR, AND METHOD FOR SUPPORTING THE THUMB OF A PATIENT AFTER BASAL JOINT ARTHROPLASTY (Attorney's Docket No. FENTON-22 PROV).

The three (3) above-identified patent applications are hereby incorporated herein by reference.

### FIELD OF THE INVENTION

This invention relates to novel orthopedic medical devices for locking an elongated element (e.g., a suture) to a bone anchor in order to reposition bone, soft tissue, or other anatomy, and to a novel sling suspension system for supporting the thumb of a patient after basal joint arthroplasty.

The novel orthopedic medical devices of the present invention may, alternatively, relate to tenodesis applications, or other soft tissue-to-bone attachment applications, in which conventional screw anchors (or other types of anchors), or other implantable medical devices, are augmented with novel suture attachment mechanisms that obviate the need to tie knots in the suture when attaching the suture to a bone anchor (or to another implantable medical device) or to tissue.

### BACKGROUND OF THE INVENTION

Degenerative osteoarthritis of the first metacarpal joint of the thumb (sometimes referred to as “the basal joint”) is a common debilitating condition, especially in middle-aged women. Research shows that 25% of all women and 8% of all men in their fifties complain of pain in the base of the thumb, secondary to basal joint osteoarthritis. Basal joint arthritis can cause substantial pain, digit weakness and loss of functionality.

In severe cases of basal joint arthritis, basal joint reconstruction may be required. In general, basal joint reconstruction involves trapezial excision (sometimes referred to as “basal joint arthroplasty”), which is required for pain relief, and subsequent soft tissue reconstruction in order to restore proper balance, alignment, longitudinal length and function to the thumb.

Unfortunately, such soft tissue reconstruction has traditionally involved harvesting tendons which are subsequently transferred and re-directed via drill holes in the thumb metacarpal in order to restore proper balance, alignment, longitudinal length and function to the thumb. However, this means harvesting, and thereby sacrificing, all or some of one or more existing, functioning tendons. In addition, such soft tissue reconstruction typically requires temporary pinning of the anatomy, which can give rise to pin tract complications such as infection, nerve damage and neuroma formation.

As a result, a new approach is needed for effecting basal joint reconstruction after performing basal joint arthroplasty in order to alleviate basal joint arthritis.

In addition to the foregoing, in many situations, anatomical objects (e.g., bone, soft tissue, prostheses, etc.) need to be attached to one another. And in many situations, it may be desirable to effect this attachment with suture. However, it can sometimes be problematic to tie knots in the suture so as to fix the anatomical objects relative to one another. In addition, it can sometimes be problematic to form through-holes in bone to secure the suture to the bone. Therefore, a new approach is needed for making knotless fixations of anatomical objects without having to drill through-holes in bone.

### SUMMARY OF THE INVENTION

The present invention comprises the provision and use of novel suture-securing mechanisms that enable the tightening and locking (e.g., cinching) of suture to an anchor or other implantable medical device without the need to tie knots in the suture in order to adjustably secure the suture to the anchor or other implantable medical device.

In one preferred form of the invention, there is provided suture-locking washers which are mounted to bone anchors, wherein the suture-locking washers comprise suture-securing mechanisms.

The present invention also comprises the provision and use of bone anchors and novel suture-locking washers for use as part of a novel sling suspension system which may be used to effect basal joint reconstruction after performing basal joint arthroplasty in order to alleviate basal joint arthritis.

More particularly, in one preferred form of the invention, the invention comprises suture-locking washers comprising suture-securing mechanisms, wherein the suture-locking washers are utilized in combination with bone anchors to form a novel sling suspension system which can be used to join the thumb metacarpal to the index metacarpal, whereby to enable tightening and locking (e.g., cinching) of a sling (e.g., a suture) of the sling suspension system so as to support the thumb of a patient after basal joint arthroplasty. The present invention eliminates the specific complications and increased morbidity associated with tendon harvesting and transfer procedures, and also eliminates the complications and increased morbidity associated with those procedures that require temporary pin fixation or drill holes for tendon or suture passage. The novel sling suspension system of the present invention provides secure and stable support for the thumb of a patient after performing basal joint arthroplasty so as to alleviate basal joint arthritis, and is fast and simple to use, since the suture-securing mechanism of the suture-locking washer eliminates the need to tie knots in the suture in order to secure the suture to a bone anchor.

In one preferred form of the invention, there is provided a sling suspension system for supporting the thumb of a patient after basal joint arthroplasty, the sling suspension system comprising:
an index metacarpal anchor;
a thumb metacarpal anchor; and
a sling (e.g., a suture) for securing the thumb metacarpal anchor to the index metacarpal anchor;
the index metacarpal anchor comprising at least one bone-engaging element for securing the index metacarpal anchor to bone and a sling-engaging element for capturing the sling to the index metacarpal anchor;
the thumb metacarpal anchor comprising at least one bone-engaging element for securing the thumb metacarpal anchor to bone and a sling-engaging element for capturing the sling to the thumb metacarpal anchor;
the sling comprising an elongated body having a first anchor-engaging element for securing the sling to the sling-engaging element of the index metacarpal anchor, and a second anchor-engaging element for securing the sling to the sling-engaging element of the thumb metacarpal anchor;
wherein the at least one sling-engaging element of the thumb metacarpal anchor comprises at least one suture-locking washer for adjustably securing the sling to the thumb metacarpal anchor.

In another preferred form of the invention, there is provided a method for supporting the thumb of a patient after basal joint arthroplasty, the method comprising:
securing a first end of a sling to an index metacarpal anchor;
positioning the index metacarpal anchor in the index metacarpal;
adjustably securing the second end of the sling to a thumb metacarpal anchor;
inserting the thumb metacarpal anchor into the thumb metacarpal; and
tightening the sling (e.g., a suture) in one direction through an adjustable suture-securing mechanism of the thumb metacarpal anchor so as to fixedly connect the thumb metacarpal to the index metacarpal.

In one preferred form of the invention, the suture-securing mechanism of the thumb metacarpal anchor is provided via a suture-locking washer which is secured to the thumb metacarpal anchor.

In another preferred form of the invention, there is provided a method for alleviating basal joint arthritis, the method comprising:
excising the trapezium of a patient;
securing a first end of a sling (e.g., a suture) to an index metacarpal anchor;
positioning the index metacarpal anchor in the index metacarpal;
adjustably securing the second end of the sling to a thumb metacarpal anchor;
inserting the thumb metacarpal anchor into the thumb metacarpal; and
tightening the sling by pulling a free end of the sling relative to a suture-securing mechanism of the thumb metacarpal anchor so that the second end of the sling is fixedly secured to the thumb metacarpal anchor, whereby the sling fixedly connects the thumb metacarpal to the index metacarpal.

In one preferred form of the invention, the suture-securing mechanism of the thumb metacarpal anchor is provided via a suture-locking washer which is secured to the thumb metacarpal anchor.

In another preferred form of the invention, the suture-locking washer (which carries the suture-securing mechanism) is formed integral with the thumb metacarpal anchor.

In another preferred form of the invention, the suture-locking washer (which carries the suture-securing mechanism) is mounted to a bone anchor (which may be used as the thumb metacarpal anchor, or as the index metacarpal anchor, or as an anchor for other locations within the body) or to another implantable medical device.

And in another preferred form of the invention, the suture-securing mechanism may be incorporated directly into a bone anchor (which may be used as the thumb metacarpal anchor, or as the index metacarpal anchor, or as an anchor for other locations within the body) or into another implantable medical device.

And in another form of the invention, a novel method and apparatus is provided for effecting knotless fixations of anatomical objects.

In another form of the invention, there is provided a sling suspension system for supporting the thumb of a patient after basal joint arthroplasty, the sling suspension system comprising:
an index metacarpal anchor;
a thumb metacarpal anchor; and
a sling for securing said thumb metacarpal anchor to said index metacarpal anchor;
said index metacarpal anchor comprising at least one bone-engaging element for securing said index metacarpal anchor to an index metacarpal, and a sling-engaging element for attaching said sling to said index metacarpal anchor;
said thumb metacarpal anchor comprising at least one bone-engaging element for securing said thumb metacarpal anchor to a thumb metacarpal, and a sling-engaging element for attaching said sling to said thumb metacarpal anchor;
wherein at least one of said sling-engaging element of said thumb metacarpal anchor and said sling-engaging element of said index metacarpal anchor comprises a sling-securing mechanism which is configured to provide one-way movement of said sling relative to said sling-securing mechanism.

In another form of the invention, there is provided a method for supporting the thumb of a patient after basal joint arthroplasty, said method comprising:
providing a sling suspension system, said sling suspension system comprising:
   an index metacarpal anchor;
   a thumb metacarpal anchor; and
   a sling for securing said thumb metacarpal anchor to said index metacarpal anchor;
   said index metacarpal anchor comprising at least one bone-engaging element for securing said index metacarpal anchor to an index metacarpal, and a sling-engaging element for attaching said sling to said index metacarpal anchor;
   said thumb metacarpal anchor comprising at least one bone-engaging element for securing said thumb metacarpal anchor to a thumb metacarpal, and a sling-engaging element for attaching said sling to said thumb metacarpal anchor;
   wherein at least one of said sling-engaging element of said thumb metacarpal anchor and said sling-engaging element of said index metacarpal anchor comprises a sling-securing mechanism which is configured to provide one-way movement of said sling relative to said sling-securing mechanism;
attaching said sling to said sling-engaging element of said index metacarpal anchor and attaching said sling to said sling-engaging element of said thumb metacarpal anchor;
positioning said index metacarpal anchor in an index metacarpal;
inserting said thumb metacarpal anchor into a thumb metacarpal; and
moving said sling through said sling-securing mechanism so as to shorten the length of said sling extending between said index metacarpal anchor and said thumb metacarpal anchor so as to fixedly connect the thumb metacarpal to the index metacarpal.

In another form of the invention, there is provided apparatus for securing a first anatomical object to a second anatomical object, said apparatus comprising:
a body having an opening; and
a tubular structure disposed in said opening, said tubular structure comprising a side wall defining a passageway, a portion of said side wall of said tubular structure projecting into said passageway so as to provide one-way movement of a filament through said passageway.

In another form of the invention, there is provided a method for securing a first anatomical object to a second anatomical object, said method comprising:
providing apparatus for securing the first anatomical object to the second anatomical object, said apparatus comprising:
   a body having an opening; and
   a tubular structure disposed in said opening, said tubular structure comprising a side wall defining a passageway, a portion of said side wall of said tubular structure projecting into said passageway so as to provide one-way movement of a filament through said passageway;
passing a filament through said passageway;
associating one end of said filament with the second anatomical object and associating said body with the first anatomical object; and
moving said filament through said passageway so as to shorten the length of said filament extending between the first anatomical object and the second anatomical object so as to fixedly connect the first anatomical object to the second anatomical object.

In one form of the invention, there is provided apparatus for adjustably securing an object to a washer body, the apparatus comprising:
a washer body having a central opening;
a first passageway extending through the washer body on a first side of the central opening and a second passageway extending through the washer body on a second, opposite side of the central opening; and
a first suture-securing structure disposed in the first passageway and a second suture-securing structure disposed in the second passageway, wherein each of the first suture-securing structure and the second suture-securing structure comprise a side wall defining a suture passageway, a portion of the side wall projecting into the passageway so as to provide one-way movement of a suture through the suture passageway.

In another form of the invention, there is provided a method for securing a first anatomical object to a second anatomical object, the method comprising:
providing an apparatus for securing the first anatomical object to the second anatomical object, the apparatus comprising a first anchor body, a second anchor body and a suture connecting the first anchor body to the second anchor body, wherein a length of the suture extending between the first anchor and the second anchor is adjustable;
positioning the first anchor body in the first anatomical structure so as to secure the first anchor body to the first anatomical structure;
positioning the second anchor body in the second anatomical structure so as to secure the second anchor body to the second anatomical structure; and
adjusting the suture so as to shorten the length of suture extending between the first anatomical object and the second anatomical object so as to fixedly connect the first anatomical object to the second anatomical object.

### BRIEF DESCRIPTION OF DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing the bone structure of the right hand of a human;

FIG. 2 is a schematic view showing a basal joint reconstruction using the novel sling suspension system of the present invention;

FIG. 3 is a schematic view showing a novel sling suspension system formed in accordance with the present invention, wherein the novel sling suspension system comprises an index metacarpal anchor with a suture secured thereto, and a thumb metacarpal anchor with a suture adjustably secured thereto, wherein the suture is adjustably secured to the thumb metacarpal anchor via a suture-locking washer;

FIGS. 4-11 are various views of selected portions of the novel sling suspension system shown in FIG. 3;

FIG. 12 is a flattened view of selected portions of the suture-securing mechanism of the suture-locking washer shown in FIGS. 4-11;

FIGS. 13-15 are schematic views showing another novel suture-securing mechanism formed in accordance with the present invention, wherein the suture-securing mechanism comprises a plurality of protrusions which facilitate mounting the suture-securing mechanism to a suture-locking washer;

FIG. 16 is an perspective schematic view of the suture-securing mechanism shown in FIGS. 13-15;

FIGS. 17-23 are schematic views of another novel suture-locking washer formed in accordance with the present invention;

FIGS. 24-27 are various schematic sectional views of the suture-locking washer of FIGS. 17-23;

FIGS. 28-30 are schematic views of a deployment assembly for deploying the novel sling suspension system of the present invention;

FIGS. 31 and 32 are schematic views of another deployment assembly for deploying the novel sling suspension system of the present invention;

FIGS. 33-53 are schematic views illustrating another novel suture-locking washer and thumb metacarpal anchor formed in accordance with the present invention;

FIGS. 54-57 are schematic views illustrating another novel suture-locking washer and thumb metacarpal anchor formed in accordance with the present invention;

FIGS. 58-61 are schematic views illustrating how the suture-securing mechanisms of the present invention may be mounted within the suture-locking washers;

FIGS. 62-65 are various schematic views of a component which may be incorporated into a suture-locking washer, wherein the component incorporates a suture-securing mechanism;

FIG. 66 is a schematic view showing another novel sling suspension system formed in accordance with the present invention;

FIGS. 67-78 are schematic views showing a novel suture-locking washer configured for attachment to a bone anchor;

FIGS. 79-84 are schematic views showing the novel suture-locking washer of FIGS. 67-78 being threaded onto an anchor;

FIGS. 85-92 are schematic views showing another novel suture-locking washer configured for attachment to a bone anchor;

FIGS. 93-100 are schematic views showing a novel suture securing system comprising the novel suture-locking washer of FIGS. 67-78 mounted to a first anchoring element, and one or more sutures being used to connect the suture-locking washer of FIGS. 67-78 to a second anchoring element;

FIGS. 101-103 are schematic views showing an augment suture and the novel suture securing system of the present invention;

FIG. 104 is a schematic view showing another novel system formed in accordance with the present invention and useful for effecting a surgical repair; and

FIGS. 105-109 are schematic views showing exemplary surgical repairs performed in accordance with the present invention.

### DETAILED DESCRIPTION OF THE PREFERRED EMBODIMENTS

### The Human Hand

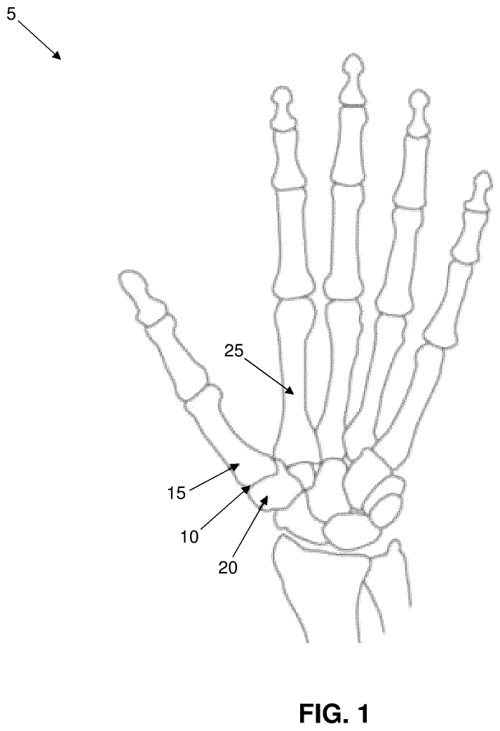

Looking first at FIG. 1, there is shown the right hand 5 of a human. As seen in the figure, the basal joint 10 is formed at the junction of the thumb metacarpal 15 and the trapezium 20. Note also the location of the adjacent index metacarpal 25.

As noted above, many people suffer from significant basal joint arthritis and would benefit from basal joint reconstruction in order to alleviate the basal joint arthritis.

### Basal Joint Reconstruction

The present invention provides a new approach for effecting basal joint reconstruction after performing basal joint arthroplasty in order to alleviate basal joint arthritis.

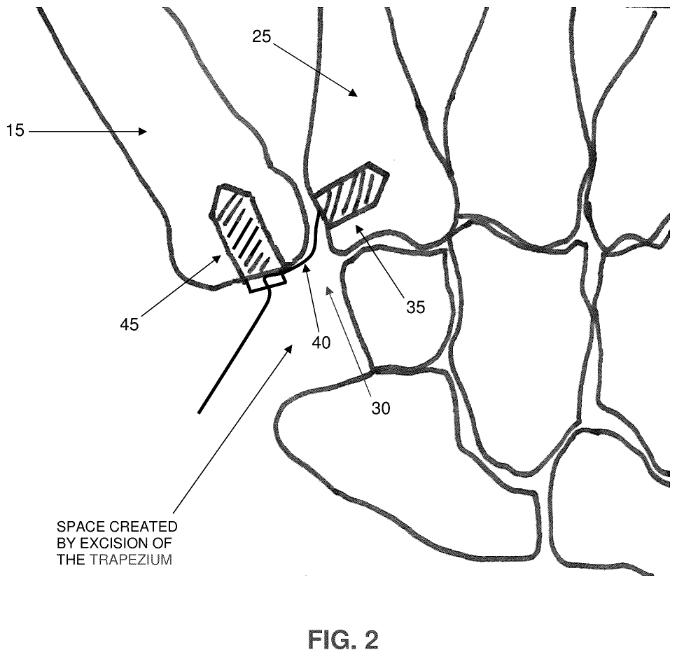

More particularly, and looking now at FIG. 2, the present invention comprises the provision and use of a novel sling suspension system 30 that joins the thumb metacarpal to the index metacarpal so as to support the thumb of a patient after basal joint arthroplasty.

As will hereinafter be discussed in further detail, and still looking at FIG. 2, novel sling suspension system 30 generally comprises an index metacarpal anchor 35 having a sling (e.g., a suture) 40 fixedly secured thereto, and a thumb metacarpal anchor 45 having sling (e.g., a suture) 40 adjustably secured thereto.

And as will hereinafter be discussed, novel sling suspension system 30 may be used in a procedure to alleviate basal joint arthritis, wherein:
the trapezium of a patient is excised;
the index metacarpal anchor is positioned in the index metacarpal;
the thumb metacarpal anchor is positioned in the thumb metacarpal; and
the sling is tightened by pulling a free end of the sling relative to a suture-securing mechanism of the thumb metacarpal anchor so that the sling is fixedly secured to the thumb metacarpal anchor, whereby the sling fixedly connects the thumb metacarpal to the index metacarpal.

The novel sling suspension system of the present invention provides secure and stable support for the thumb of a patient after performing basal joint arthroplasty in order to alleviate basal joint arthritis, and is relatively fast and simple to implement.

More particularly, the present invention comprises the provision and use of a suture-securing mechanism which may be used in concert with one or more bone anchors for effecting basal joint reconstruction after performing basal joint arthroplasty to alleviate basal joint arthritis.

In one form of the present invention, the invention comprises the provision and use of a novel sling suspension system comprising a knotless sling attachment that can be used to join the thumb metacarpal to the index metacarpal in order to support the thumb of a patient after basal joint arthroplasty. The present invention eliminates the specific complications and increased morbidity associated with tendon harvesting and transfer procedures, as well as the specific complications and increased morbidity associated with those procedures that require temporary pin fixation or drill holes for tendon or suture passage.

In one preferred form of the invention, the novel sling suspension system comprises at least one suture-locking washer for use with a bone anchor, wherein the at least one suture-locking washer comprises at least one suture-securing mechanism to enable tightening and securing (e.g., cinching) of a suture (i.e., the “sling”) once appropriate anchors (e.g., an index finger screw anchor, a thumb screw anchor, etc.) are positioned in the anatomy so as to provide stable support for the thumb of a patient after performing basal joint arthroplasty, whereby to alleviate basal joint arthritis. The novel sling suspension system of the present invention is relatively fast and simple to implement.

### The Novel Sling Suspension System Utilizing Novel Suture-Locking Washers

As noted above, many people suffer from significant basal joint arthritis and would benefit from basal joint reconstruction to alleviate the basal joint arthritis.

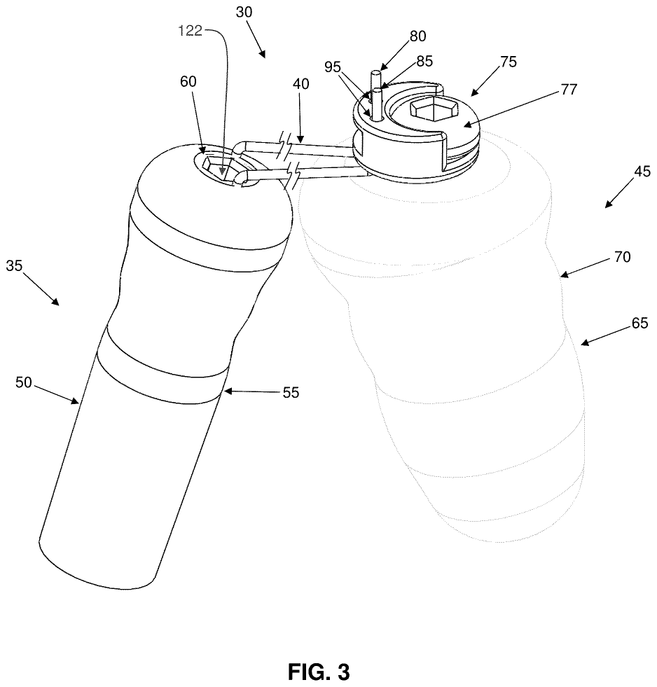

FIG. 3 shows a novel sling suspension system 30 for disposition in and between an index finger and a thumb of a human hand.

Sling suspension system 30 generally comprises index metacarpal anchor 35 for deployment in index metacarpal 25, thumb metacarpal anchor 45 for deployment in thumb metacarpal 15, and sling 40 (e.g., a suture) for securing thumb metacarpal anchor 45 to index metacarpal anchor 35, whereby to support the thumb of a patient after basal joint arthroplasty has been performed in order to alleviate basal joint arthritis. See FIG. 3. Although sling 40 will hereinafter generally be discussed in the context of a suture “sling”, it should be appreciated that sling 40 may comprise substantially any elongated flexible element, and is not intended to be limited to suture. In addition, it should also be appreciated that sling 40 may comprise structure in addition to an elongated flexible element (e.g., a suture). By way of example but not limitation, sling 40 may comprise a hammock-like structure mounted to one or more elongated flexible elements (e.g., sutures), such that the one or more elongated flexible elements (e.g., sutures) may be mounted to index metacarpal anchor 35 and thumb metacarpal anchor 45, and the hammock-like structure may engage and support portions of thumb metacarpal 15.

Index metacarpal anchor 35 is intended to be deployed in (e.g., screwed into or pushed into) a drill hole (i.e., a bone hole) formed at or near the base of index metacarpal 25. See FIG. 2. Alternatively, index metacarpal anchor 35 may comprise a self-tapping anchor, in which case the bone hole is formed during delivery of the bone anchor into the index metacarpal. Index metacarpal anchor 35 comprises a body 50 having at least one bone-engaging element 55 (e.g., screw threads, ribs, etc.) (see FIG. 3) formed thereon for engaging the side wall of the bone hole, i.e., for engaging index metacarpal 25. In this way, index metacarpal anchor 35 can be secured to index metacarpal 25. Index metacarpal anchor 35 further comprises a sling-engaging element 60 (e.g., a recessed eyelet) (see FIG. 3) for capturing sling 40 to index metacarpal anchor 35.

Thumb metacarpal anchor 45 is intended to be deployed in (e.g., screwed into or pushed into) a drill hole (i.e., a bone hole) formed at or near the base of thumb metacarpal 15. See FIG. 2. Alternatively, thumb metacarpal anchor 45 may comprise a self-tapping anchor, in which case the bone hole is formed during delivery of the bone anchor into the thumb metacarpal. Thumb metacarpal anchor 45 comprises a body 65 having at least one bone-engaging element 70 (e.g., screw threads, ribs, etc.) (see FIG. 3) formed thereon for engaging the side wall of the bone hole, i.e., for engaging thumb metacarpal 15. In this way, thumb metacarpal anchor 45 can be secured to thumb metacarpal 15. Thumb metacarpal anchor 45 further comprises a suture-locking washer 75 (i.e., a sling-engaging element) for mounting to thumb metacarpal anchor 45 and for adjustably gripping and securing sling 40 to thumb metacarpal anchor 45 as will hereinafter be discussed in further detail. In one preferred form of the invention, suture-locking washer 75 comprises a body having a hole formed therein (see FIG. 7) for mounting the suture-locking washer to thumb metacarpal anchor 45. In one preferred form of the invention, suture-locking washer 75 is rotatably secured to thumb metacarpal anchor 45 so that suture-locking washer 75 is able to traverse (i.e., rotate) 360 degrees around the longitudinal axis of body 65 of thumb metacarpal anchor 45. By way of example but not limitation, suture-locking washer 75 is rotatably mounted to thumb metacarpal anchor 45 with a screw 77 (see FIG. 3) which passes through a hole 78 (see FIG. 7) formed in suture-locking washer 75 and into thumb metacarpal anchor 45.

Sling 40 secures thumb metacarpal anchor 45 to index metacarpal anchor 35, whereby to provide, in combination with thumb metacarpal anchor 45 and index metacarpal anchor 35, a sling suspension system for supporting the thumb of a patient after basal joint arthroplasty has been performed to alleviate basal joint arthritis. To this end, in one preferred form of the invention, sling 40 comprises an elongated flexible body (e.g., a suture) having a first end 80 and a second end 85. See FIG. 3. And in one preferred form of the invention, sling 40 includes an anchor-engaging element (not shown), e.g., a knot or looped section of the sling, for securing sling 40 to index metacarpal anchor 35 such that the sling is attached to index metacarpal anchor 35. Thus, in one preferred form of the invention, sling 40 comprises first end 80 and second end 85 which engage suture-locking washer 75 (which is itself mounted to thumb metacarpal anchor 45), and an intermediate portion of the elongated flexible body (e.g., the suture) which engages index metacarpal anchor 35. See FIG. 3.

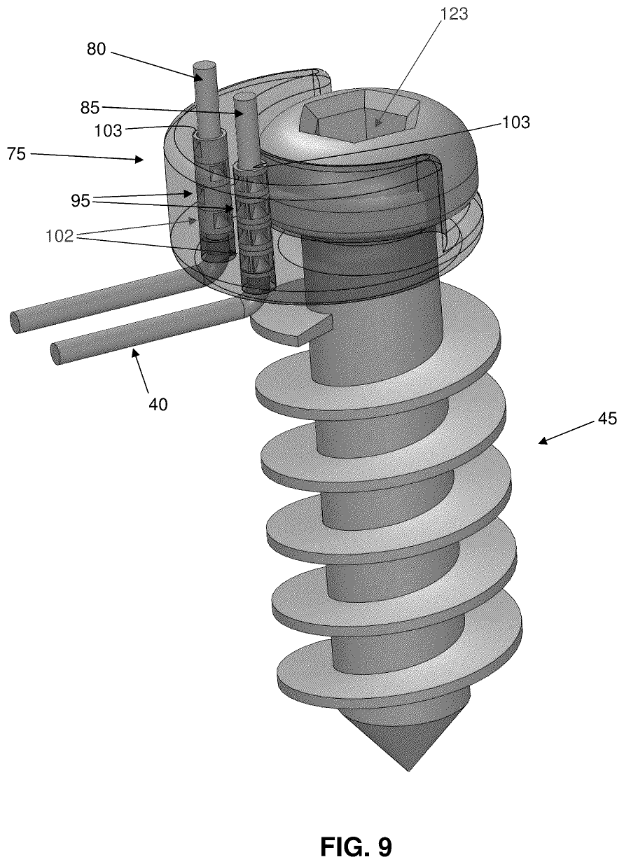

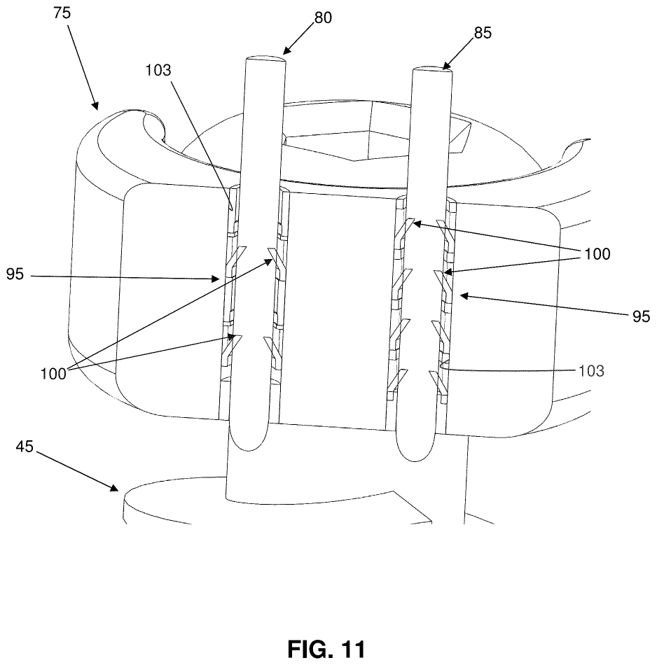

Suture-locking washer 75 comprises one or more adjustable suture-securing mechanisms 95 (see FIGS. 9-11) for securing sling 40 to suture-locking washer 75 of thumb metacarpal anchor 45 such that the sling is adjustably attached to thumb metacarpal anchor 45, as will hereinafter be discussed.

The elongated body of sling 40 may comprise a flexible filament (e.g., a suture, a rope, a web, a tape, etc.), or some other flexible elongated structure, i.e., substantially any flexible elongated structure which will suffice for the intended purpose, either man-made (e.g., biocompatible polymers, metals, etc.) or biologic (e.g., cross-linked collagen, cross-linked hydrogel, chitosan, etc.), and/or composite structures. In one preferred form of the invention, sling 40 comprises a suture. Where sling 40 comprises a suture, it should be appreciated that the suture can be a hollow or solid core braided suture, a monofilament suture, etc. It should also be appreciated that, if desired, sling 40 may be constructed and configured so that it is able to slide with respect to itself, e.g., such as where it has been passed through itself.

As shown in FIGS. 2 and 3 and discussed above, sling suspension system 30 comprises index metacarpal anchor 35 and thumb metacarpal anchor 45, both of which are fixedly secured to bone. In one preferred form of the invention, index metacarpal anchor 35 and thumb metacarpal anchor 45 both comprise screw anchors that engage the bone of a metacarpal and remain affixed to a metacarpal as the associated sling 40 (which is coupled to index metacarpal anchor 35 and thumb metacarpal anchor 45) is tightened and secured (i.e., cinched) to thumb metacarpal anchor 45. It should be appreciated that bone anchors having alternative bone-engaging mechanisms (e.g., ribs as opposed to screw threads, or expanding bodies, etc.) may alternatively (or additionally) be utilized, as will be apparent to those skilled in the art in view of the present disclosure.

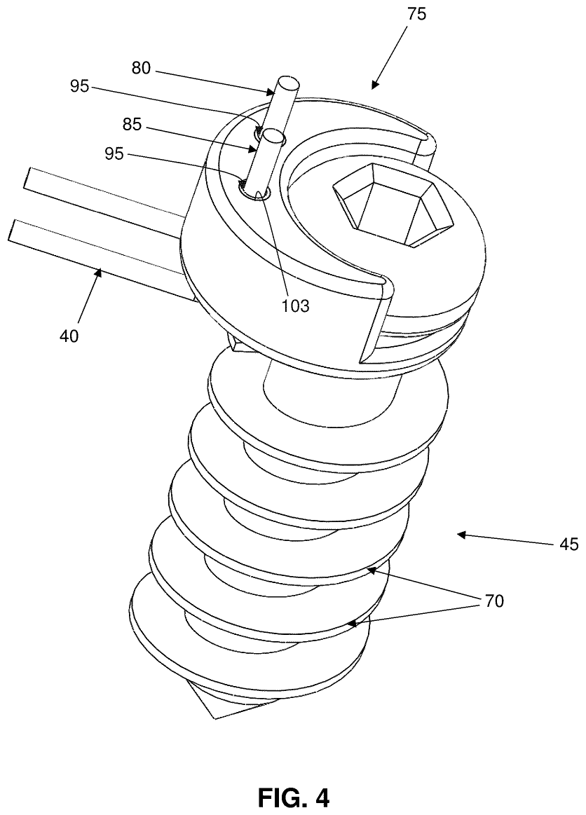

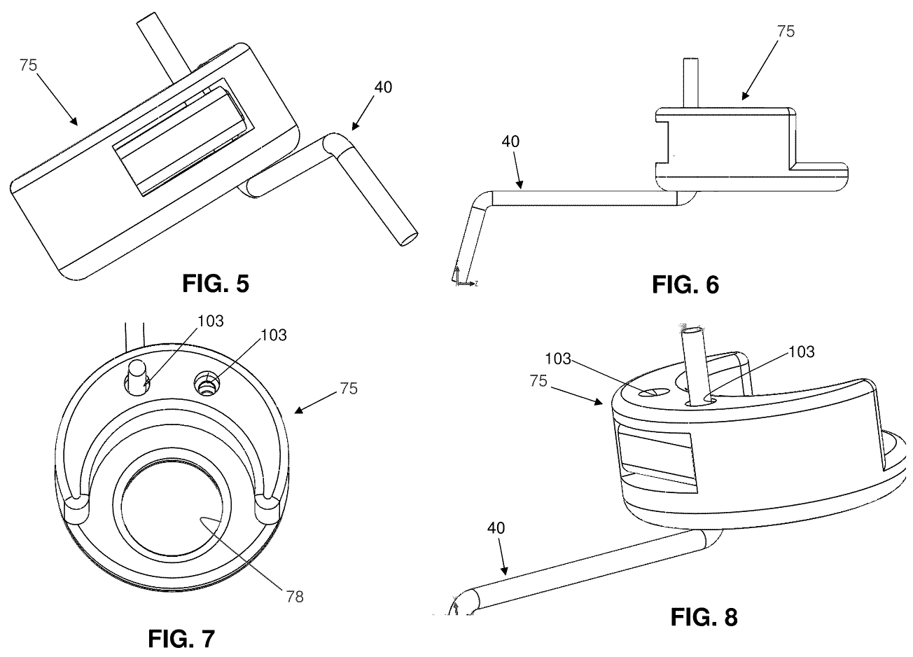

As shown in FIGS. 2-9, sling 40 preferably comprises two suture strands (preferably high-strength, high-density polyethylene suture or the equivalent) which are looped or tied (or otherwise secured) to the base (i.e., the proximal end) of index metacarpal anchor 35. Alternatively, if desired, sling 40 may comprise a single strand of suture which is looped through (or otherwise secured to) the base (i.e., the proximal end) of index metacarpal anchor 35, e.g., such as by looping the suture through sling-engaging element 60 (e.g., a recessed eyelet). The free ends 80, 85 of sling 40 are threaded through suture-securing mechanism 95 of suture-locking washer 75. Suture-locking washer 75 is mounted to thumb metacarpal anchor 45 which, in one preferred form of the invention, comprises a screw anchor. It should be appreciated that, if desired, suture-locking washer 75 may, alternatively and/or additionally, be coupled to index metacarpal anchor 35. However, due to the clinical ease of pulling the sling relative to the thumb metacarpal anchor 45, in the preferred form of the present invention, suture-locking washer 75 is secured to thumb metacarpal anchor 45 as shown in FIG. 3.

### The Suture-Securing Mechanism

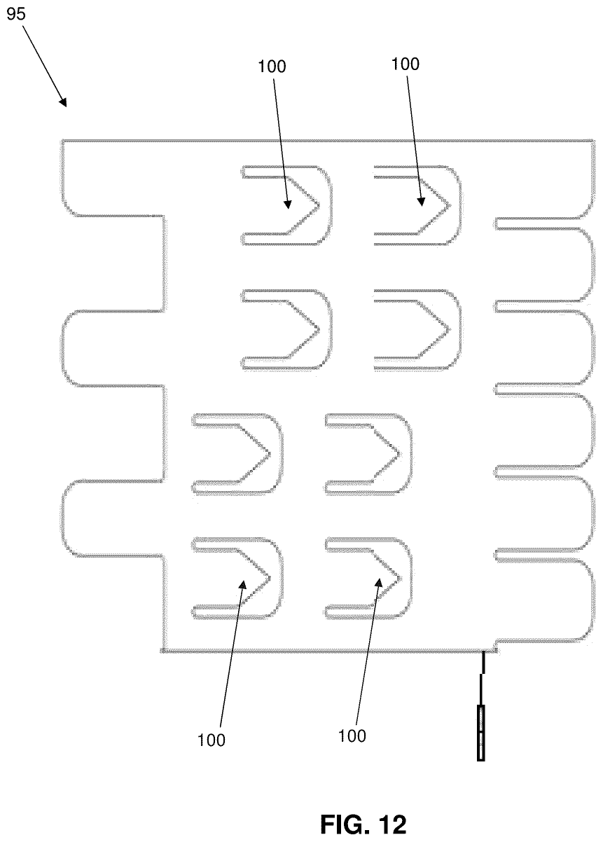

Suture-locking washer 75 is mounted to the base (i.e., the proximal end) of thumb metacarpal anchor 45, and comprises the aforementioned at least one suture-securing mechanism 95. See FIGS. 9-11. Suture-securing mechanism 95 is shown in greater detail in FIGS. 9-12. In one preferred form of the invention, two suture-securing mechanisms 95 are utilized, each of which allows the passage and attachment of a free end 80, 85 of sling 40 to suture-locking washer 75, as will hereinafter be discussed in further detail. Once index metacarpal anchor 35 is secured to the index metacarpal, and thumb metacarpal anchor 45 is positioned in the thumb metacarpal, the free end(s) 80, 85 of sling 40 are retracted (i.e., pulled, or “cinched”) as the thumb is positioned relative to the index finger. Suture-securing mechanisms 95 (shown in flattened form in FIG. 12) engage the first and second ends 80, 85 of sling 40 with suture-holding elements 100 (e.g., barbs) in order to allow one-way passage of first and second ends 80, 85 of sling 40 through suture-securing mechanisms 95, whereby to enable one-way tightening (i.e., cinching) of sling 40 upon positioning of the thumb metacarpal 15 relative to index metacarpal 25. As this occurs, suture-securing mechanisms 95 prevent sling 40 from sliding in the opposite direction of tightening, thereby preventing slipping of sling 40 and affixedly attaching sling 40 (and thus thumb metacarpal 15 and index metacarpal 25) into stable position during healing of the newly reconstructed basal joint.

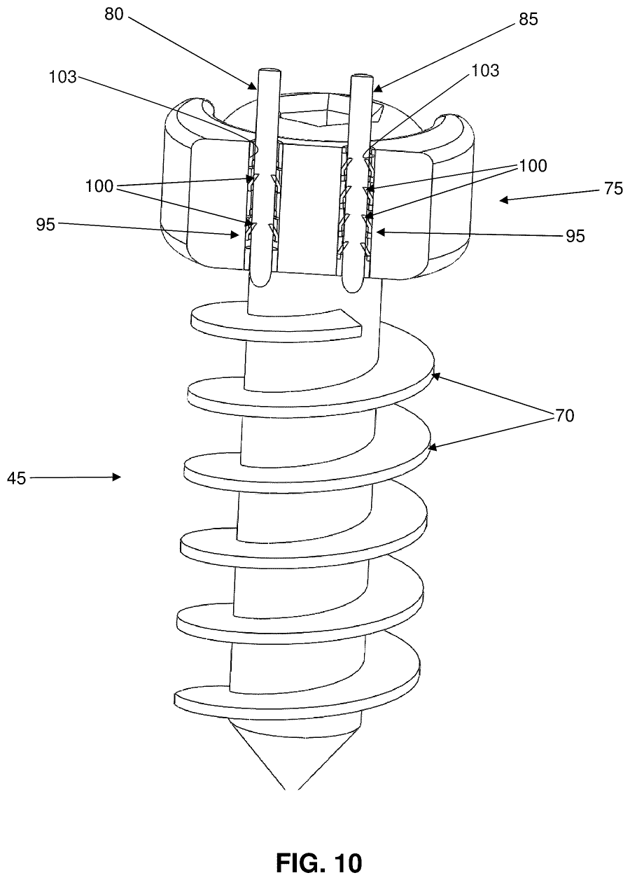

The suture-securing mechanisms 95 shown in FIGS. 9-12 generally comprise tubular structures 102 (FIG. 9) which are laser-cut into (or otherwise formed into) a pattern including suture-holding elements (barbs or teeth) 100 (FIG. 10) which are bent or positioned inwardly when the tube is assembled so as to form the suture-securing mechanism 95, whereby to engage sling 40.

In one preferred form of the invention, and looking now at FIG. 12, tubular structures 102 start as a flat sheet of material, have suture-holding elements (barbs or teeth) 100 laser cut into the sheet of material, and then the sheet of material is bent into a tubular structure (i.e., tubular structure 102) so as to provide the complete suture-securing mechanisms 95.

Tubular structures 102 of suture-securing mechanisms 95 are fixedly secured in holes 103 formed in suture-locking washer 75, whereby to fix suture-securing mechanisms 95 relative to suture-locking washer 75. See FIG. 9.

In one preferred form of the invention, tubular structures 102 of suture-securing mechanisms 95 are press-fit into holes 103.

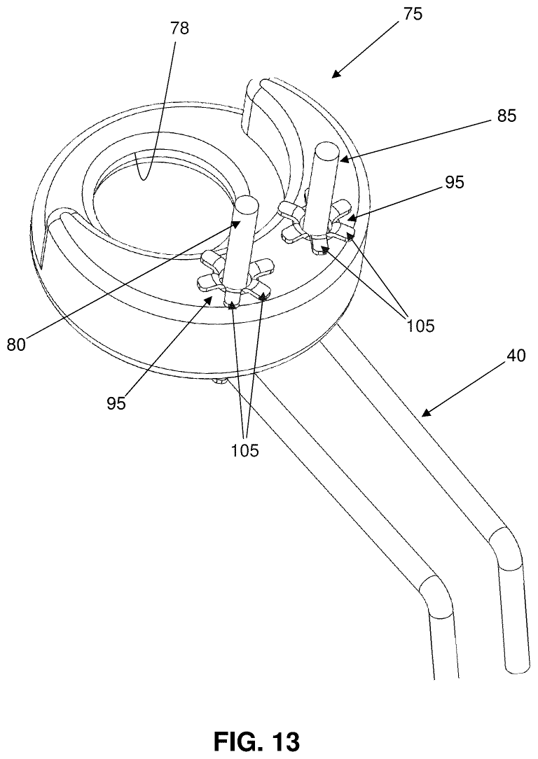

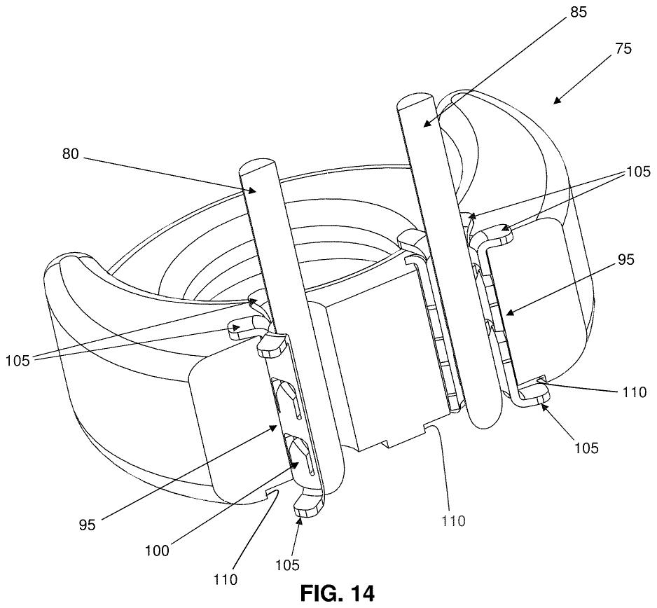

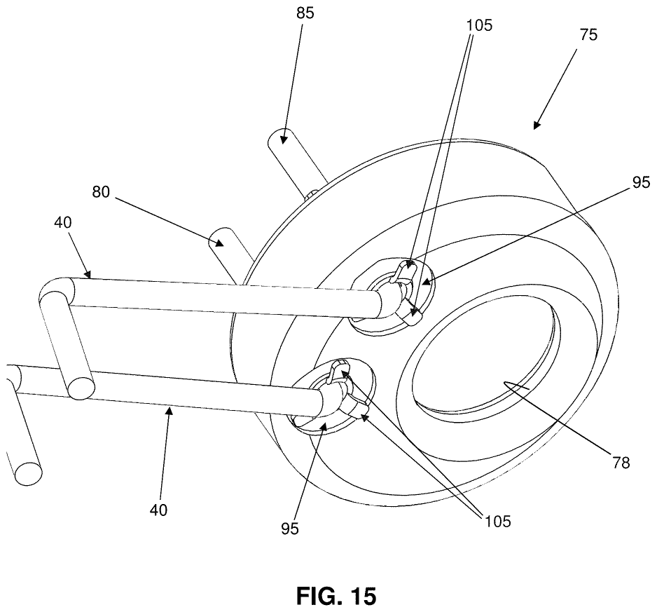

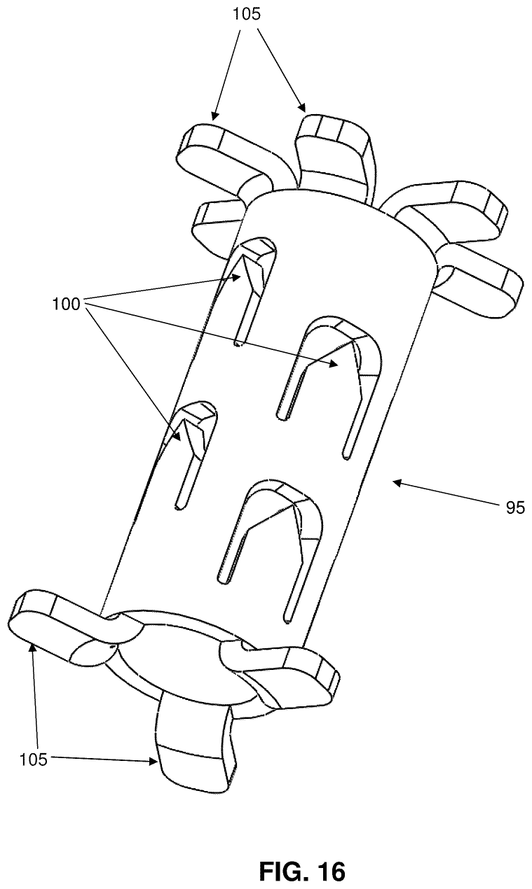

In another preferred form of the invention, and looking now at FIGS. 13-16, suture-securing mechanisms 95 may also comprise protrusions 105 that may be positioned to engage the outer surfaces of suture-locking washer 75 so as to maintain the position of the suture-securing mechanisms 95 relative to suture-locking washer 75. If desired, one or both ends of holes 103 may be provided with a counterbore 110 (FIG. 14) for receiving protrusions 105.

In one preferred form of the invention, suture-securing mechanisms 95 may comprise two “rows” and four “columns” (i.e., relative to the longitudinal axis of the suture-securing mechanism 95) of suture-holding elements (barbs or teeth) 100 that are offset from one another in order to prevent compressing sling 40 between the “points” of opposing barbs or teeth 100. Four columns are preferable in order to allow pre-positioning of the barbs or teeth 100 inwardly. Fewer or more rows of barbs or teeth 100 may be used, provided that the desired pull force is achieved and the height of anchor washer 75 (which is partially determined by the length of the suture-securing mechanism) is small enough to limit the profile of the thumb metacarpal anchor 45 (including the suture-locking washer 75 mounted thereto) from extending above the base of thumb metacarpal 15 upon deployment of thumb metacarpal anchor 45. If desired, suture-securing mechanisms 95 may comprise fewer columns with longer barb or teeth widths so that the barbs or teeth 100 do not fracture or weaken upon deflecting, and/or barbs or teeth 100 may be pre-positioned inwardly so as to allow engagement of sling 40. Such a pattern of barbs or teeth 100 increases the pull force while limiting damage to sling 40.

It should also be appreciated that alternative shapes and/or numbers of barbs or teeth 100 may be utilized to engage sling 40.

### Use of the Novel Sling Suspension System

Sling suspension system 30 may be used in the following manner to support the thumb of a patient after a basal joint arthroplasty has been performed to alleviate basal joint arthritis.
1. The patient's trapezium is excised (see FIG. 2) to alleviate the basal joint arthritis.
2. Index metacarpal 25 and thumb metacarpal 15 are prepared to receive index metacarpal anchor 35 and thumb metacarpal anchor 45, respectively. In one preferred form of the invention, a bone hole is drilled into the thumb side of index metacarpal 25, and a bone hole is drilled into the base of thumb metacarpal 15. Note that the space created by the excised trapezium allows easy access to the base of thumb metacarpal 15 and to the thumb side of index metacarpal 25.
3. Sling 40 is attached to index metacarpal anchor 35 using sling-engaging element 60 (e.g., an eyelet) of index metacarpal anchor 35 and an anchor-engaging element (e.g., a knot) of sling 40, and sling 40 is attached to suture-locking washer 75 and suture-locking washer 75 is attached to thumb metacarpal anchor 45, if they are not already in this arrangement. See FIG. 3.
4. Index metacarpal anchor 35 is secured in the bone hole formed in index metacarpal 25.
5. Thumb metacarpal anchor 45 is secured in the bone hole formed in the base of thumb metacarpal 15.
6. The free end(s) 80, 85 of sling 40 is/are tensioned (i.e., cinched), whereby to correctly position thumb metacarpal anchor 45 relative to index metacarpal anchor 35, and hence to correctly position thumb metacarpal 15 relative to index metacarpal 25, so as to provide the desired reconstructed basal joint. See FIG. 3.
7. Free end(s) 80, 85 of secured sling 45 can then be cut with an appropriate instrument if desired.

### Alternative Form of Suture-Locking Washer

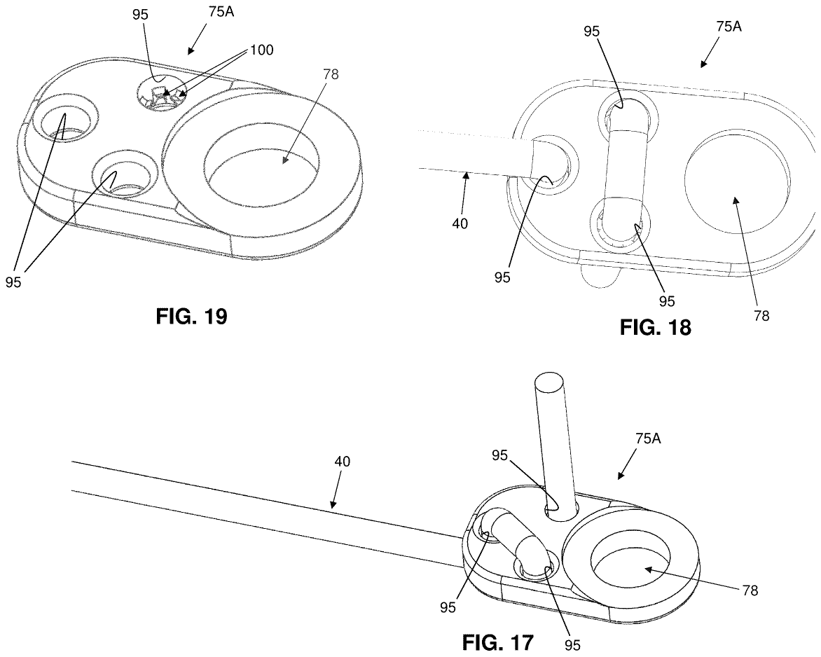

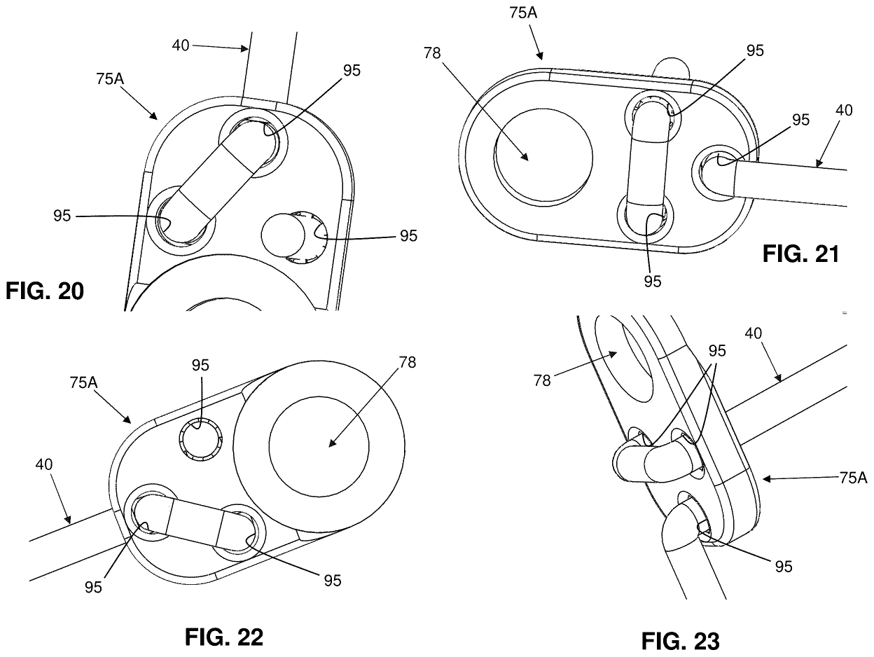

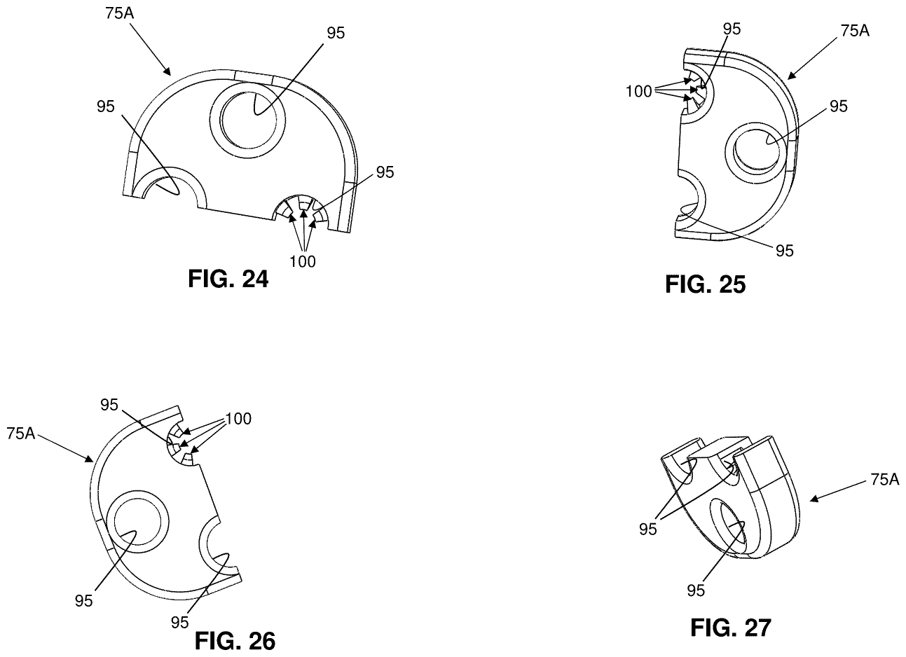

FIGS. 17-27 show an alternative suture-locking washer 75A wherein the one or more suture-securing mechanisms 95 are formed integral with the body of suture-locking washer 75A (i.e., the side walls of suture-securing mechanisms 95 are the side walls of holes 103 passing through the suture-locking washer). See FIGS. 19 and 24-26. In this form of the invention, suture-locking washer 75A comprises multiple holes 103 for sling 40 (i.e., a suture) to pass through, with at least one of the holes 103 including suture-holding elements (barbs or teeth) 100 positioned so as to allow passage of sling 40 in one direction while preventing slippage of the sling in the opposite direction. Others of holes 103 may omit barbs or teeth 100 (i.e., others of holes 103 may comprise smooth inside walls).

### Deployment Assembly

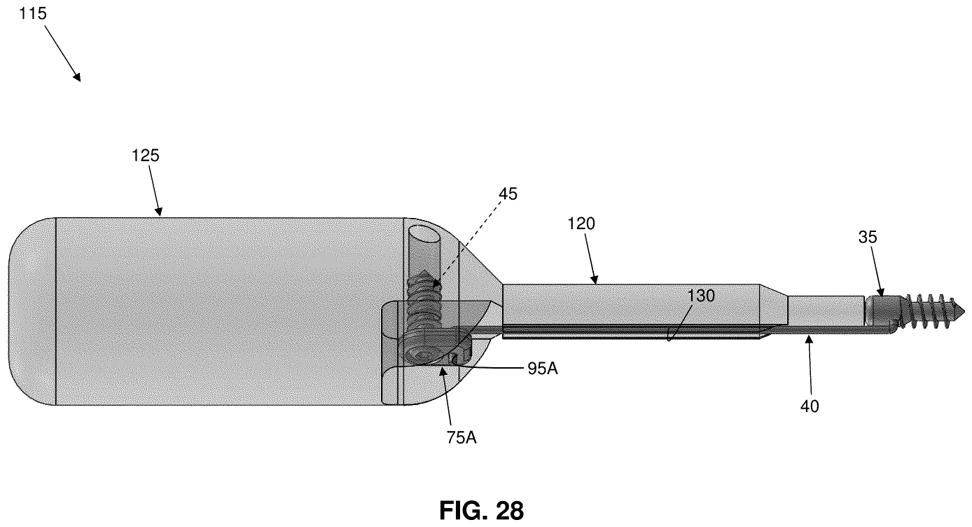

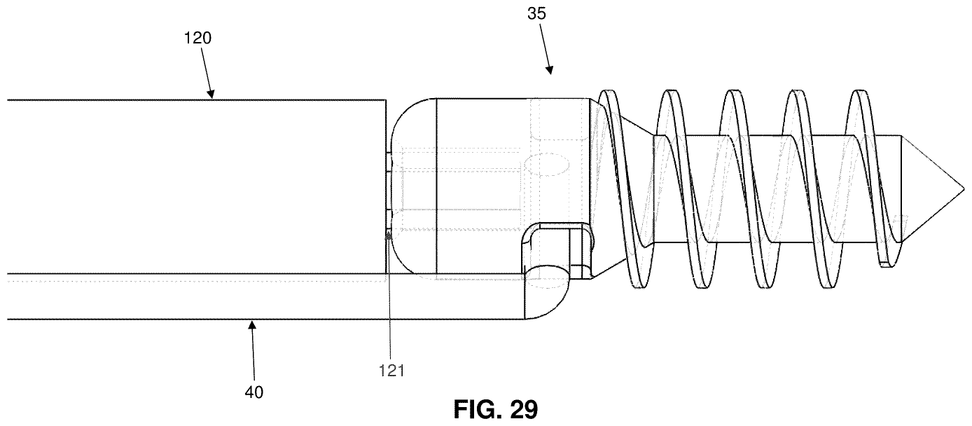

Looking now at FIGS. 28-30, there is shown a deployment assembly 115 which may be used to deploy sling suspension system 30. More particularly, deployment assembly 115 may be used to set index metacarpal anchor 35 into index metacarpal 25 (e.g., to insert index metacarpal anchor 35 into a bone hole formed in index metacarpal 25) and/or to set thumb metacarpal anchor 45 into thumb metacarpal 15 (e.g., to insert thumb metacarpal anchor 45 into a bone hole formed in thumb metacarpal 15). To this end, deployment assembly 115 preferably comprises a shaft 120 having a drive feature (e.g., a hex driver) 121 (FIG. 29) at its distal end for engaging (i) a counterpart drive feature (e.g., a hexagonal recess) 122 (FIG. 3) formed in the proximal end of index metacarpal anchor 35 so as to enable deployment assembly 115 to be used to drive index metacarpal anchor 35 into index metacarpal 25, and/or (ii) a counterpart drive feature (e.g., a hexagonal recess) 123 (FIG. 9) formed on the proximal end of thumb metacarpal anchor 45 so as to enable deployment assembly 115 to be used to drive thumb metacarpal anchor 45 into thumb metacarpal 15.

A handle 125 (FIG. 28) is provided at the proximal end of shaft 120 of deployment assembly 115.

In one exemplary form of the invention, suture-locking washer 75A is affixed to thumb metacarpal anchor 45 at the time of manufacture, so that the surgeon does not have to worry about fixing the suture-locking washer to the thumb metacarpal anchor at the time of use. See FIG. 28.

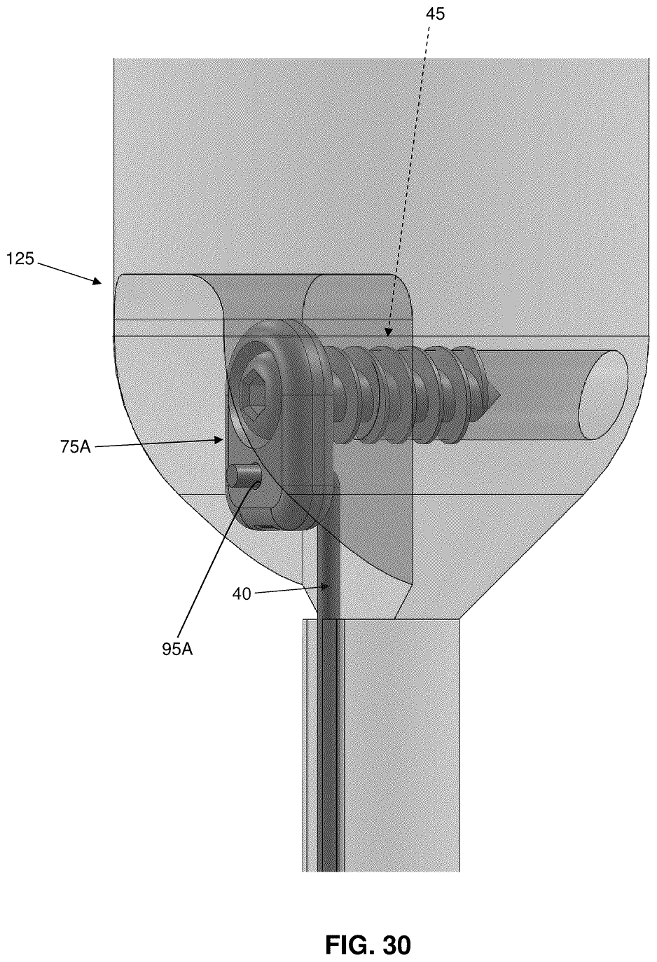

And in one exemplary form of the invention, index metacarpal anchor 35 is pre-loaded onto the distal end of shaft 120 of deployment assembly 115 at the time of system assembly, e.g., as shown in FIGS. 28 and 29. Thumb metacarpal anchor 45, with suture-locking washer 75A pre-affixed thereto, is preferably pre-loaded onto handle 125 of deployment assembly 115 (FIGS. 28 and 30). After deployment assembly 115 has been used to set index metacarpal anchor 35 into index metacarpal 25, thumb metacarpal anchor 45 (with suture-locking washer 75A pre-affixed thereto) is loaded on the distal end of shaft 120 of deployment assembly 115 and deployment assembly 115 is then used to set thumb metacarpal anchor 45 (with suture-locking washer 75A pre-affixed thereto) into thumb metacarpal 15.

Where index metacarpal anchor 35 is pre-loaded onto deployment assembly 115 at the time of system assembly, deployment assembly 115 is preferably provided with a channel 130 (FIGS. 28 and 30) that allows passage of sling 40 along shaft 120 to a suture-locking washer 75A mounted onto a thumb metacarpal anchor 45.

### Alternative Deployment Assembly

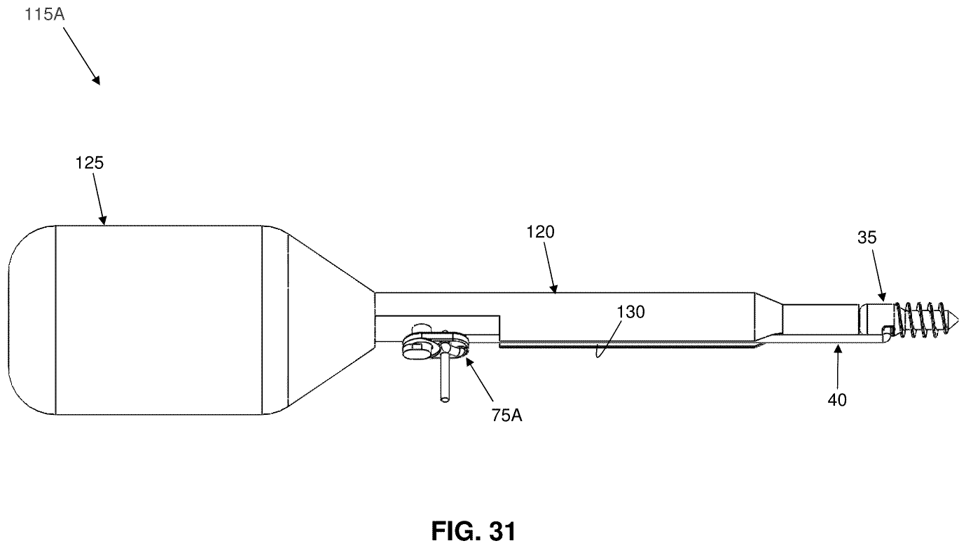

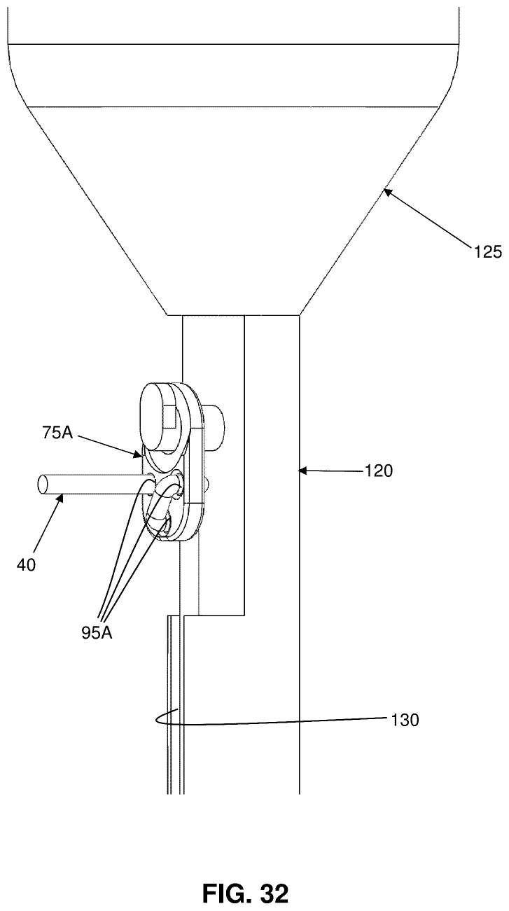

FIGS. 31 and 32 show another form of deployment assembly 115A, wherein thumb metacarpal anchor 45 is loaded onto shaft 120 of deployment assembly 115A (as opposed to thumb metacarpal anchor 45 being loaded onto handle 125 of deployment assembly 115).

### Another Suture-Locking Washer and Thumb Metacarpal Anchor

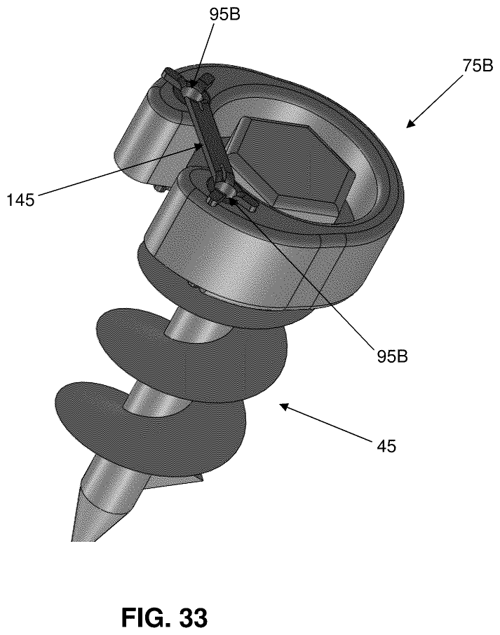

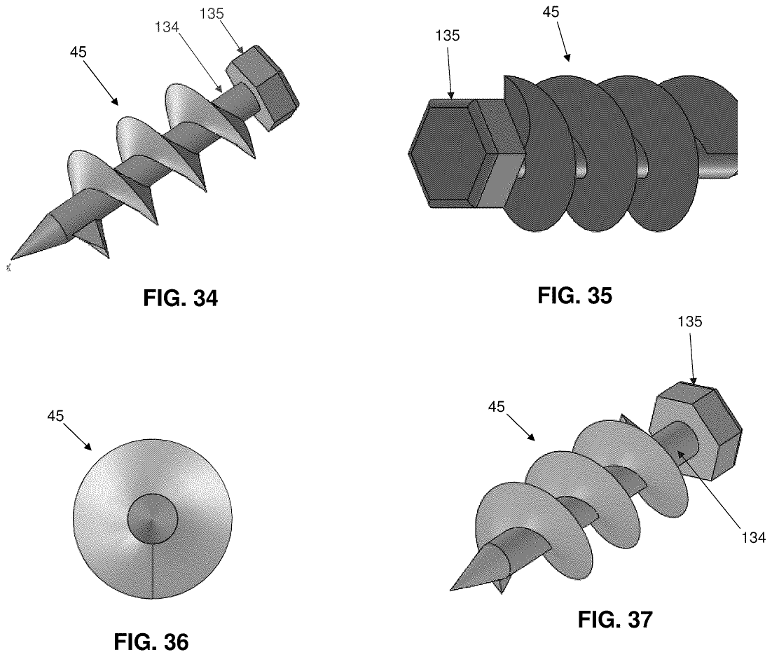

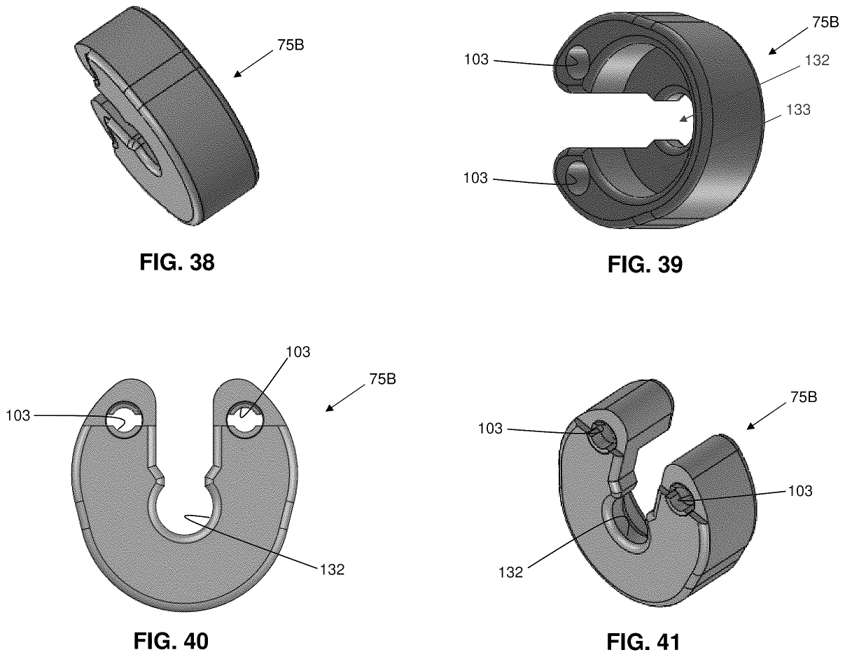

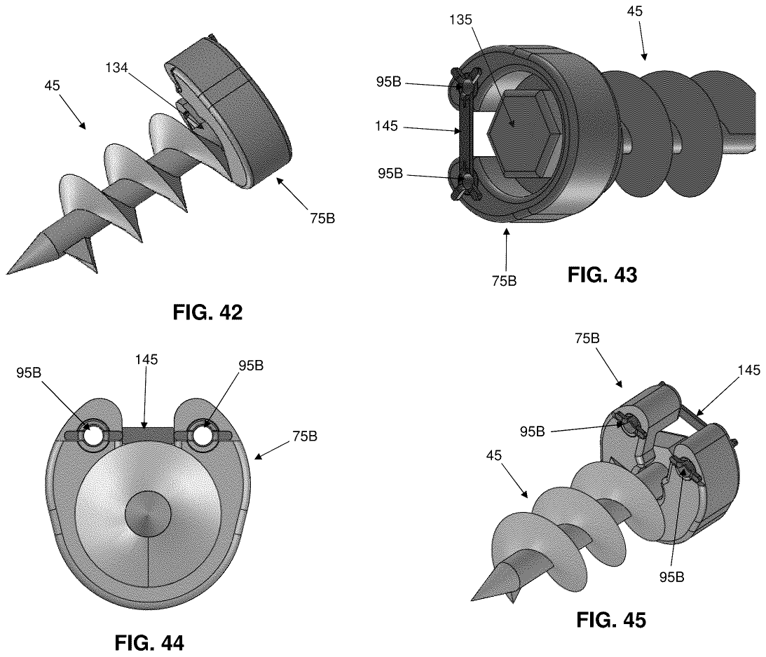

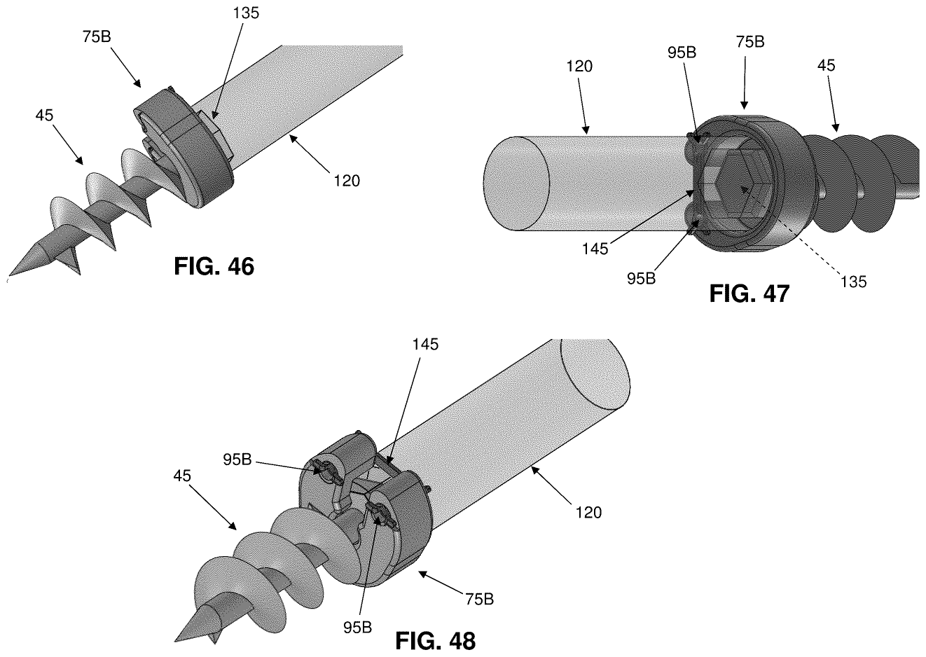

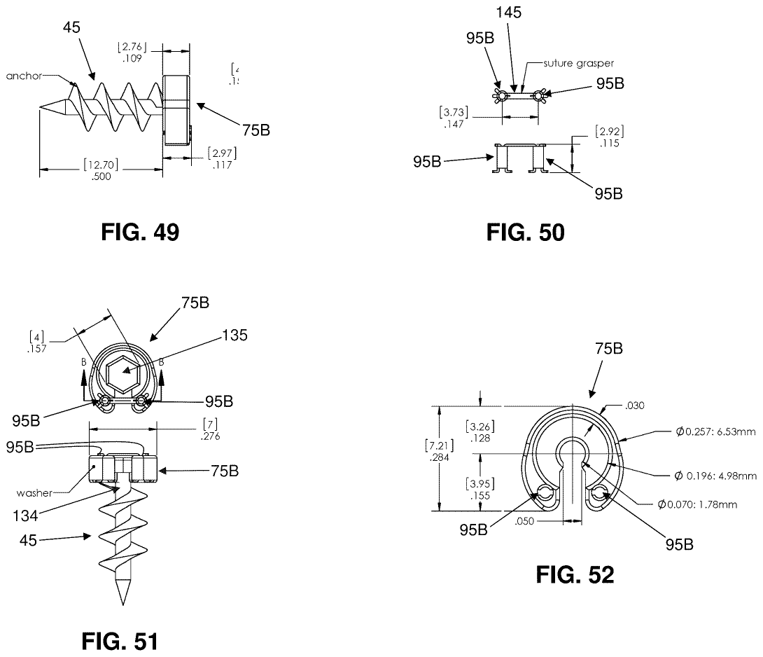

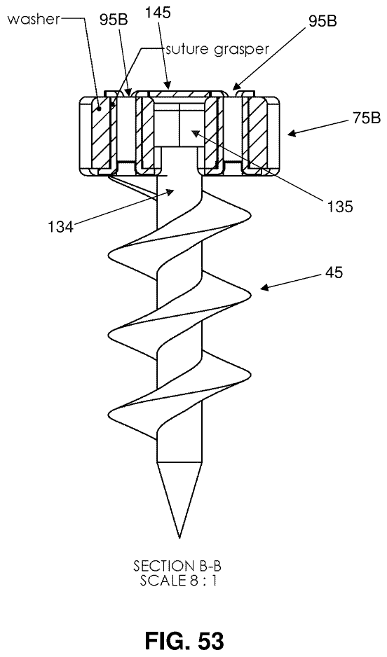

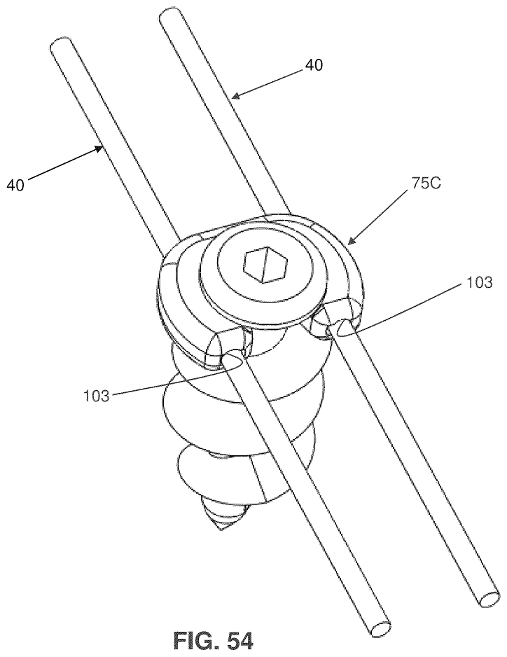

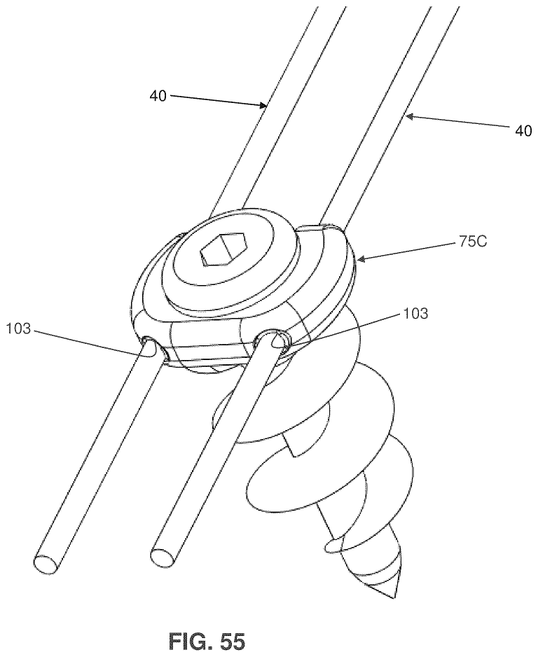

FIGS. 33-53 show still another form of suture-locking washer and thumb metacarpal anchor formed in accordance with the present invention. More particularly, in this form of the invention, there is provided a suture-locking washer 75B comprising at least two suture-securing mechanisms 95B. See FIG. 33. Suture-locking washer 75B is configured to mount to the proximal end of a screw-type thumb metacarpal anchor 45, whereby to permit the suture to be knotlessly secured to thumb metacarpal anchor 45. Anchor washer 75B is generally C-shaped (FIGS. 39-41) and preferably comprises a bore 132 and a counterbore 133, wherein bore 132 receives the shaft 134 of thumb metacarpal anchor 45 (FIG. 43) and counterbore 133 receives the head 135 of thumb metacarpal anchor 45, whereby to facilitate mounting anchor washer 75B to the proximal end of thumb metacarpal anchor 45. In this form of the invention, suture-securing mechanisms 95B preferably comprise a connecting bar 145 (see FIGS. 33 and 43) for connecting one suture-securing mechanism 95B to an adjacent suture-securing mechanism 95B, and for securing anchor washer 75B to the proximal end of thumb metacarpal anchor 45. It should be appreciated that anchor washer 75B may be secured to thumb metacarpal anchor 45 and/or sling 40 and/or deployment assembly 115 at the time of manufacture and may be sterilized as a unit prior to delivery to an end user (e.g., a surgeon).

### Suture-Locking Washer for Securing Suture Perpendicular to the Longitudinal Axis of an Anchor

In the foregoing disclosure, suture-locking washers 75, 75A and 75B are disclosed as having suture-securing mechanisms 95 mounted to the suture-locking washer such that (a) free end(s) of sling 40 (e.g., the free end(s) of a suture) can be passed through a given suture-securing mechanism 95 which is, in turn, mounted to a given suture-locking washer 75, 75A or 75B, whereby to facilitate knotless securement of the free end(s) of sling 40 to a suture-locking washer 75, 75A, 75B.

And, in the foregoing disclosure, the aforementioned suture-locking washers 75, 75A and 75B are disclosed as having one or more suture-securing mechanisms 95 disposed parallel to the longitudinal axis of an anchor to which the suture-locking washer 75, 75A and 75B is attached. See, for example, FIGS. 9, 30 and 53.

However, it should also be appreciated that, if desired, the one or more holes 103 formed in the suture-locking washer which are configured to receive the one or more suture-securing mechanisms 95 may be disposed perpendicular (rather than parallel) to the longitudinal axis of an anchor to which the suture-locking washer is mounted. Thus, in one form of the invention, suture-securing mechanisms 95 are disposed perpendicular to the longitudinal axis of an anchor to which the suture locking washer is mounted.

By way of example but not limitation, and looking now at FIGS. 54 and 55, there is shown a novel suture-locking washer 75C formed in accordance with the present invention. Suture-locking washer 75C comprises a plurality of holes 103 oriented perpendicular to the longitudinal axis of an anchor to which suture-locking washer 75C is mounted.

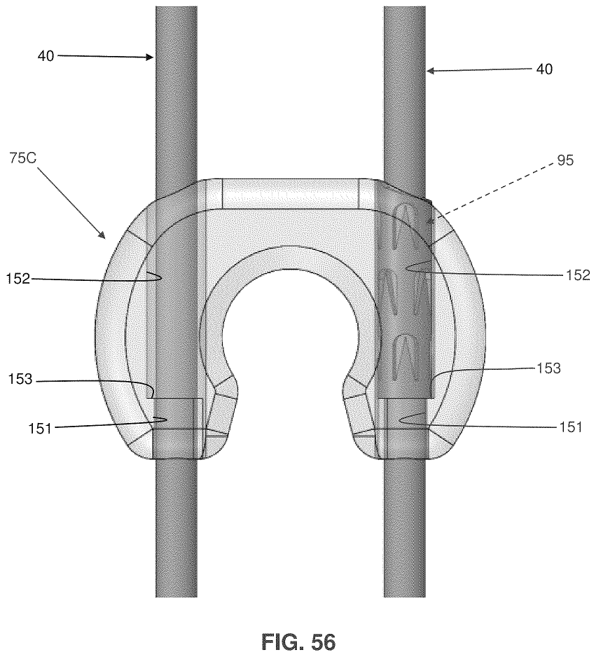

In this form of the invention, and looking now at FIG. 56, holes 103 preferably comprise stepped bores 151 and 152, which together define a shoulder 153 formed therein. Shoulder 153 provides a stop for seating a suture-securing mechanism 95 within a hole 103. Note that, if desired, similar shoulders may be provided in holes 103 of suture-locking washers 75, 75A and 75B.

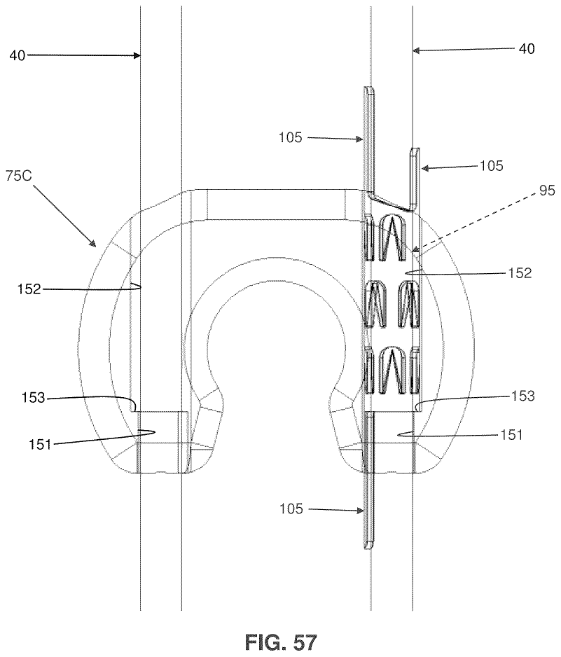

It should be appreciated that, in this form of the invention, suture-securing mechanism 95 may also comprise the aforementioned protrusions 105 for securing suture-securing mechanisms 95 to suture-locking washers 75C, i.e., in the manner previously discussed. See FIG. 57. It should also be appreciated that, in this form of the invention, shoulder 153 may be cut-away as appropriate to enable one or more protrusions 105 to extend out of bores 151.

In one form of the invention, suture-securing mechanisms 95 may be mounted to suture-locking washer 75C (or to suture-locking washers 75, 75A or 75B) before sling 40 is passed through suture-securing mechanisms 95.

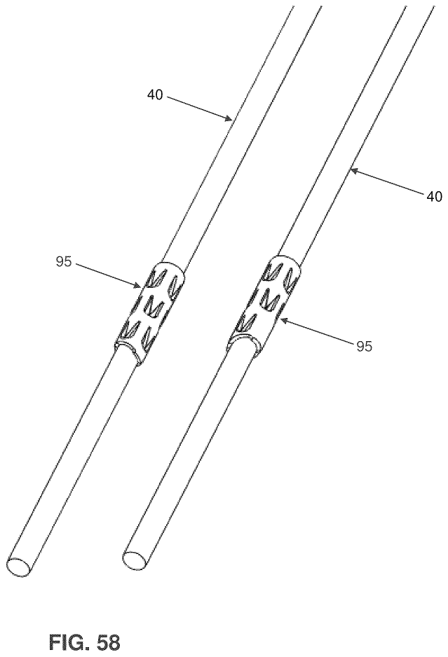

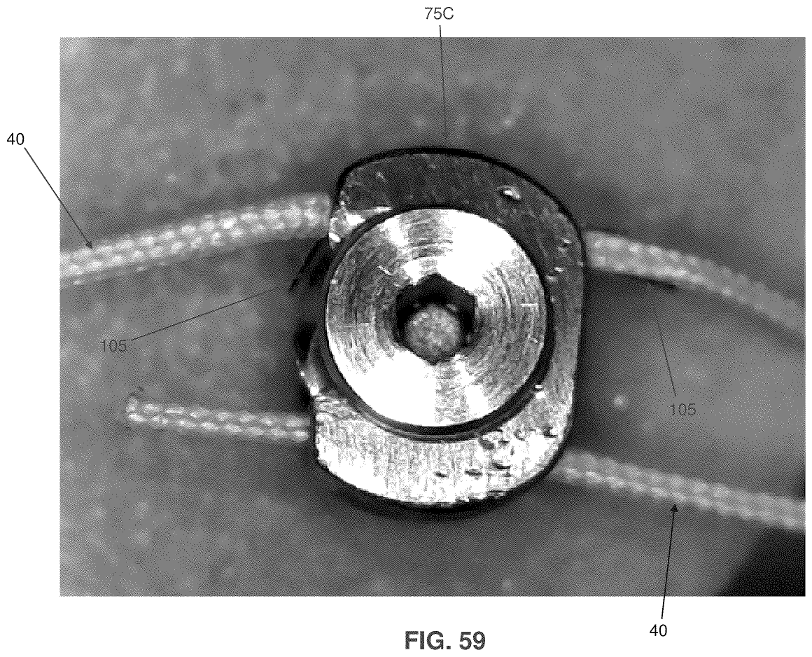

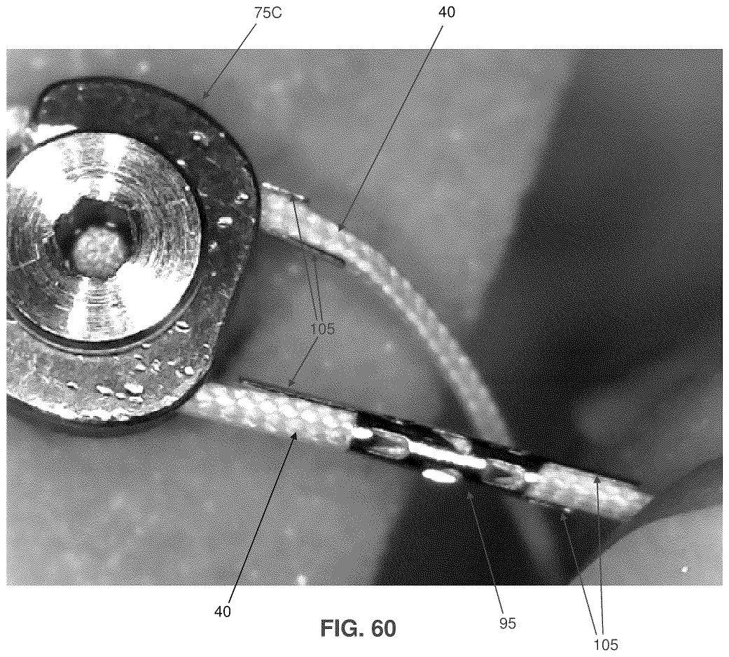

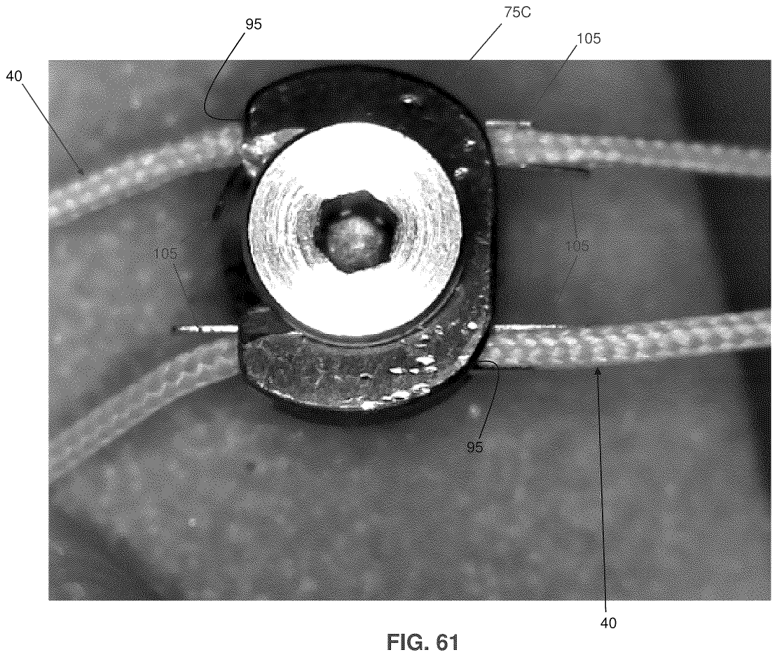

Alternatively, in another form of the invention, sling 40 may be passed through suture-securing mechanism 95 before suture-securing mechanism 95 is mounted to suture-locking washer 75C (or to suture-locking washers 75, 75A or 75B). More particularly, with this approach, suture-securing mechanisms 95 are mounted to sling 40, e.g., a suture (FIG. 58), the free ends of sling 40 (e.g., the free ends of a suture) are threaded through holes 103 in suture-locking washer 75C (FIG. 59), and then suture-securing mechanisms 95 are “towed” into place within holes 103 (FIG. 60) and secured to the suture-locking washer (FIG. 61).

### Component Which may be Incorporated Into a Suture-Locking Washer

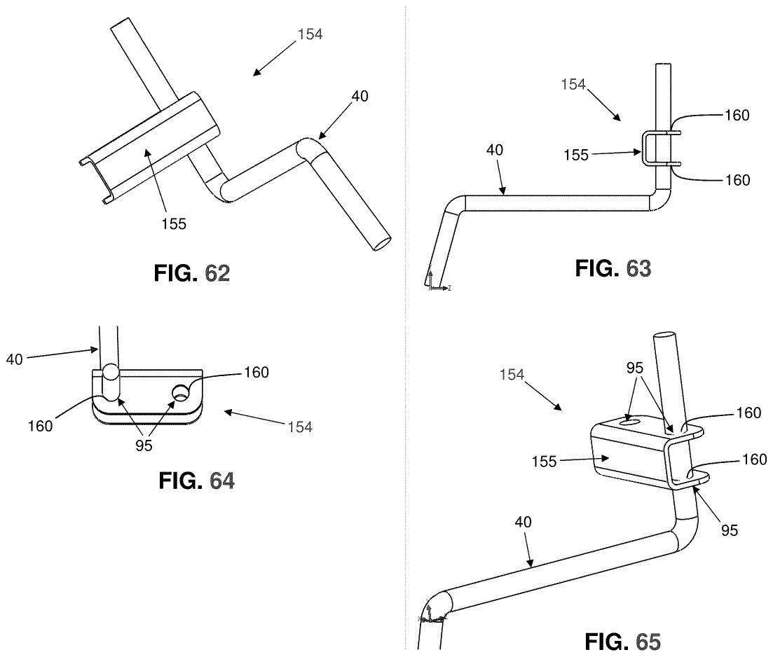

FIGS. 62-65 show a component which may be incorporated into a suture-locking washer, wherein the component incorporates a suture-securing mechanism. More particularly, in this form of the invention, there is provided a component 154 that comprises a housing 155 which includes a pair of openings 160 which receive suture-securing mechanisms 95. If desired, a suture-securing mechanism 95 may be disposed in an opening 160, or a suture-securing mechanism 95 may be mounted in, and extend between, two aligned openings 160. Furthermore, some of the openings 160 may omit a suture-securing mechanism 95 (i.e., some of the openings 160 may comprise a smooth-walled bore). Component 154 is configured so that it may be incorporated into a suture-locking washer of the sort described above, whereby to adjustably secure a sling (e.g., a suture) 40 to component 154, and hence to adjustably secure a sling (e.g., a suture) 40 to a suture-locking washer and bone anchor.

### Suture-Locking Washer Which is not Mounted to a Bone Anchor

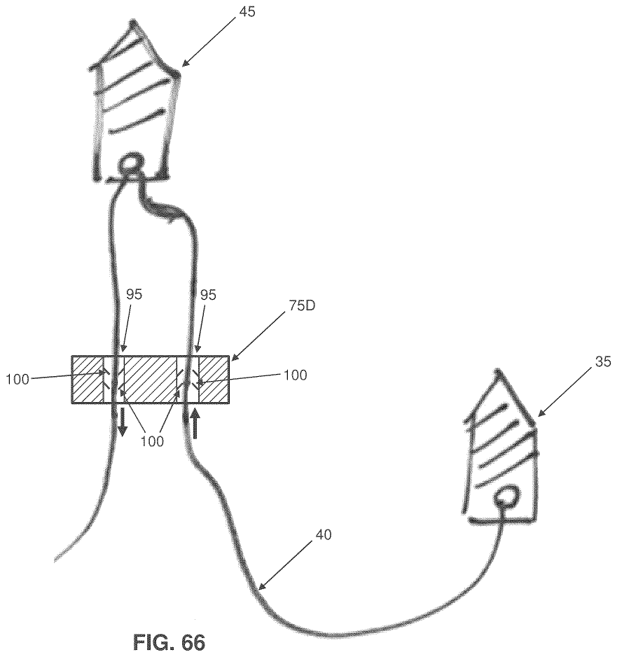

In another form of the invention, and looking now at FIG. 66, sling suspension system 30 may comprise an index metacarpal anchor 35 for deployment in index metacarpal 25, a thumb metacarpal anchor 45 for deployment in thumb metacarpal 15, and a sling 40 (e.g., a suture) for securing thumb metacarpal anchor 45 to index metacarpal anchor 35. In this form of the invention, sling 40 is secured to index metacarpal anchor 35, and sling 40 is slidable relative to thumb metacarpal anchor 45. A suture-locking washer 75D is slidably mounted onto sling 40. Suture-locking washer 75D comprises two suture-securing mechanisms 95, wherein the two suture-securing mechanisms 95 are configured to allow suture movement in opposite directions. More particularly, the suture-securing mechanism 95 receiving the portion of sling 40 extending between index metacarpal anchor 35 and thumb metacarpal anchor 45 allows suture movement toward thumb metacarpal anchor 45 but prevents suture movement toward index metacarpal anchor 35, and the portion of sling 40 extending between thumb metacarpal anchor 45 and the other suture-securing mechanism 95 allows suture movement away from thumb metacarpal anchor 45 but prevents suture movement toward thumb metacarpal anchor 45.

In this form of the invention, index metacarpal anchor 35 is disposed in index metacarpal 25, thumb metacarpal anchor 45 is disposed in thumb metacarpal 15, thumb metacarpal 15 is positioned in the appropriate location relative to index metacarpal 25, and then the free end of sling 40 is pulled as suture-locking washer 75D is advanced towards thumb metacarpal anchor 45 until suture-locking washer 75D is seated against thumb metacarpal 15. The locking engagement of suture-locking washer 75D with sling 40 keeps thumb metacarpal 15 locked against movement relative to index metacarpal 25.

### Alternative Suture-Locking Washer

It has been found that, with the aforementioned suture-locking washers 75, 75B, 75C the general “horseshoe” shape of the suture-locking washer provides both benefits and, potentially, at least one a downside.

Specifically, the “horseshoe” shape of suture-locking washers 75, 75B, 75C facilitates quick and easy attachment to a bone anchor (e.g., thumb metacarpal anchor 45) due to the opening between the arms of the “horseshoe” shaped suture-locking washers 75, 75B, 75C since the opening allows the suture-locking washers 75, 75B, 75C to flex slightly to accommodate the shaft of the bone anchor (see FIG. 45) within the central bore of the suture-locking washer (e.g., central bore 132 of suture-locking washer 75B). One benefit of such a construction is that the bone anchor can be implanted into the anatomy, and the washer can thereafter be passed over the head of the bone anchor and mounted thereto.

However, as a result of this construction, it has also been found that “horseshoe” shaped suture-locking washers 75, 75B, 75C may inadvertently become detached from the bone anchor during, or after, the bone anchor has been deployed in the anatomy. Specifically, the opening between the arms of the “horseshoe” shape also creates a potential weakness, in that the washer may inadvertently slide off of the bone anchor after attachment/mounting of the washer to the bone anchor. This issue may be addressed by the provision of the aforementioned connecting bar 145 provided with suture-locking washer 75B, which connecting bar acts to prevent the washer from slipping off of the head of the anchor after attachment/mounting to the head of the anchor. However, connecting bar 145 is a very small element that generally must be provided already mounted to the body of the suture-locking washer 75B (i.e., the suture-locking washer 75B must generally be provided in the form of an already-assembled anchor and washer, with connecting bar 145 already mounted to the washer). Thus, the flexibility (i.e., ability to permit the surgeon to selectively attach, or detach, the suture-locking washer from the anchor after implantation) facilitated by the aforementioned suture-locking washers 75, 75B, 75C is undermined by the provision of a connecting bar such as connecting bar 145. And, inasmuch as such a construct must be provided pre-assembled, such a construction means that the surgeon must rely on use of the anchor to which the suture-locking washer 75, 75B, 75C is already attached.

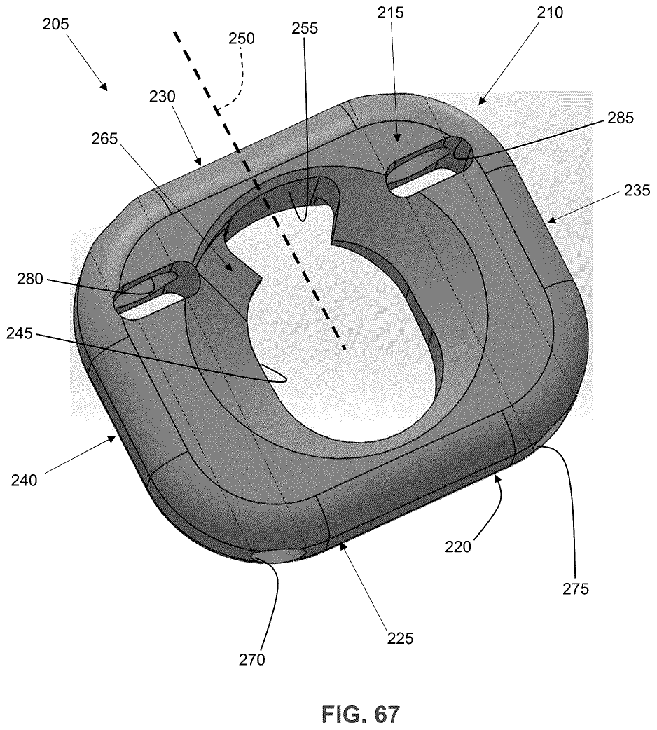

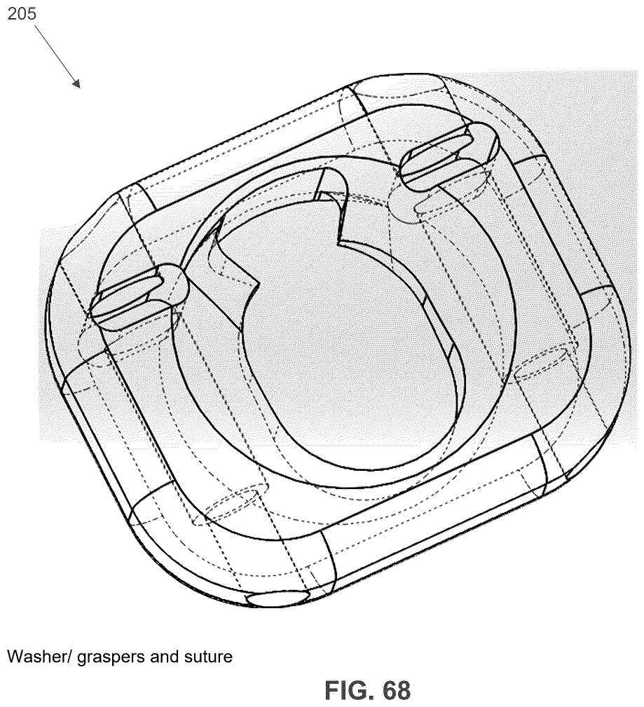

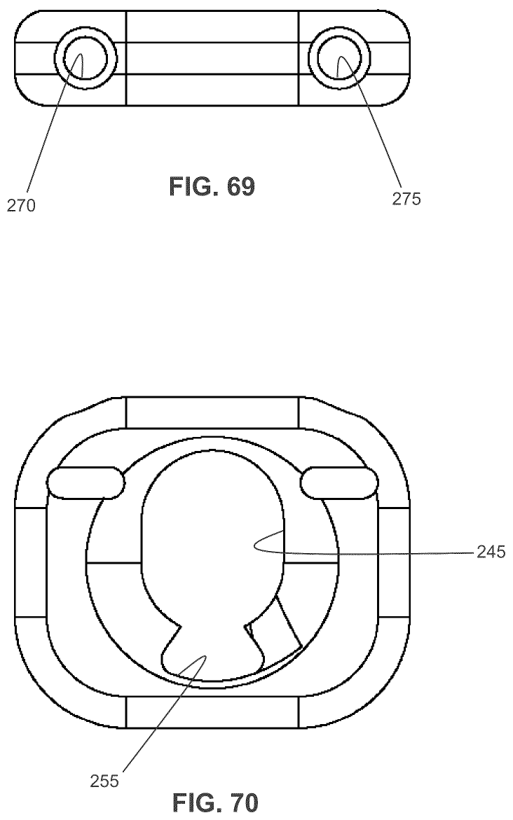

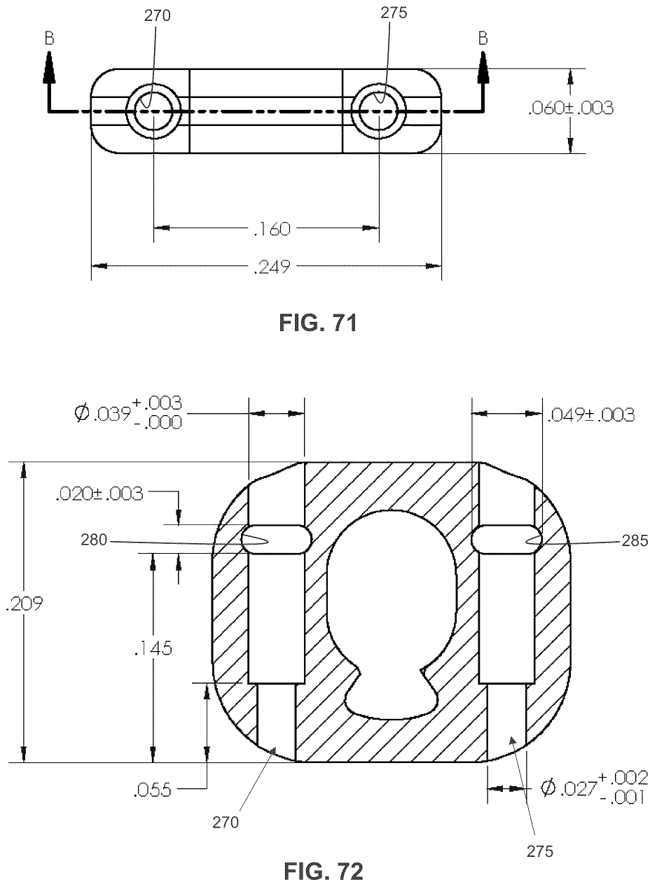

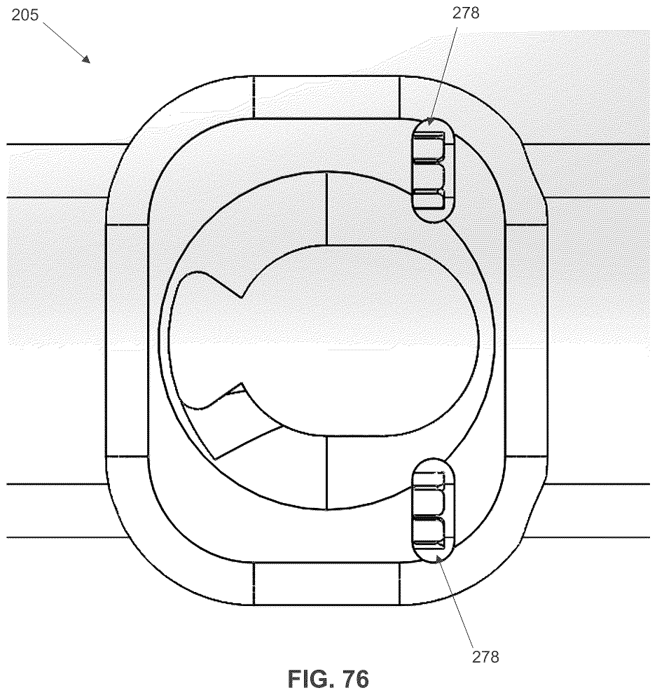

To address these limitations, and looking now at FIGS. 67-78, there is shown another novel suture-locking washer 205 formed in accordance with the present invention.

Suture-locking washer 205 generally comprises a body 210 having an upper surface 215, a lower surface 220, a front surface 225, a rear surface 230, and two side surfaces 235, 240. In a preferred form of the invention, body 210 of suture-locking washer 205 is generally rectangular in shape. However, it will be appreciated that, if desired, body 210 of suture-locking washer 205 may comprise other geometries (e.g., square, circular, etc.) which will be apparent to one of skill in the art in view of the present disclosure.

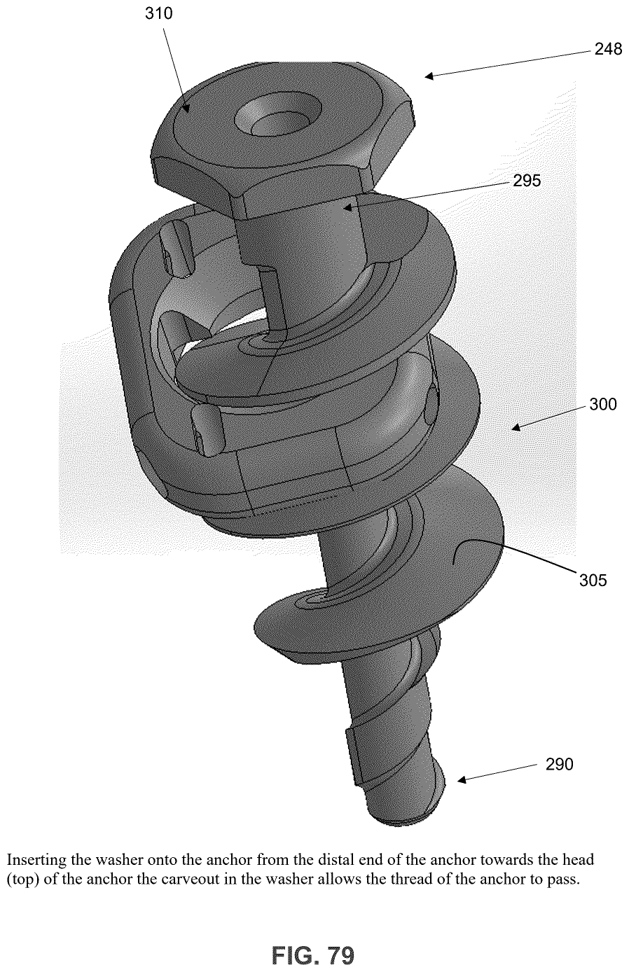

A central bore 245 is formed in body 210 extending between upper surface 215 and lower surface 220 of body 210 parallel to a central longitudinal axis 250. Central bore 245 comprises an irregular geometry inasmuch as central bore 245 is configured to receive an anchor 248 (FIG. 79) comprising a portion having a thread defining a “cork screw” style geometry, whereby to screw suture-locking washer 205 onto anchor 248, as will hereinafter be discussed in further detail.

Central bore 245 comprises a side cutout 255. A portion of body 210 in the region where it surrounds central bore 245 is formed with decreasing thickness (along the axis parallel to central longitudinal axis 250) so as to form a lead-in chamfered surface 265 in the region of side opening 255, whereby to facilitate mounting of (e.g., screwing of) suture-locking washer 205 to the head of anchor 248, as will hereinafter be discussed in further detail.

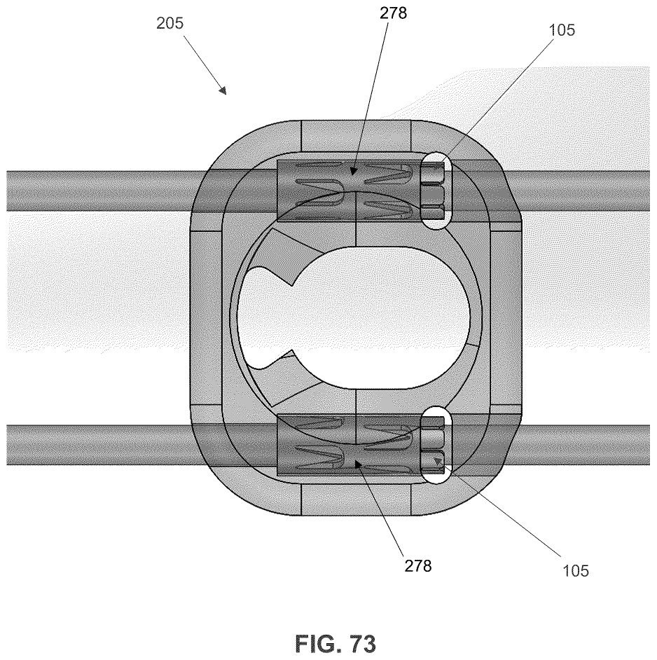

A pair of bores 270, 275 are formed in body 210 of suture-locking washer 205 extending between, and opening upon, front surface 225 and rear surface 230 (i.e., extending perpendicular to central longitudinal axis 250). Bores 270, 275 are sized to receive a suture-securing mechanism 278 (FIGS. 73-76). It should be appreciated that suture-securing mechanism 278 may comprise the aforementioned suture-securing mechanisms 95, 95B so as to permit one-way movement of suture relative to suture-locking washer 205 in the same manner as discussed above with respect to suture-locking washers 75, 75A, 75B, 75C. Alternatively and/or additionally, and as discussed in further detail below, suture-securing mechanism 278 may comprise additional features to aid in securing suture-securing mechanism 278 to body 210 of suture-locking washer 205.

If desired, a first top window 280 may be formed in upper surface 215 (and/or in lower surface 220) of body 210 extending parallel to central longitudinal axis 250 and opening on bore 275. As a result of this construction, when a suture-securing mechanism 278 is disposed in bore 270 such that an end of the suture-securing mechanism is disposed in alignment with first top window 280, a suture may be passed through the portion of bore 270 opening on rear surface 230 (perpendicular to central longitudinal axis 250), and up through first top window 280 such that the suture extends generally parallel to central longitudinal axis 250. Such a disposition of a suture may be desired where there is limited access to front surface 225 (e.g., due to the surrounding anatomy) or in situations in which the surgeon desires to cinch the suture relative to the suture-locking washer 205 by pulling the suture in a direction generally parallel to central longitudinal axis 250, etc. It should be appreciated that, if desired, a second top window 285 may be provided which is disposed such that second top window 285 opens on bore 275 in the same manner discussed above with respect to first top window 280.

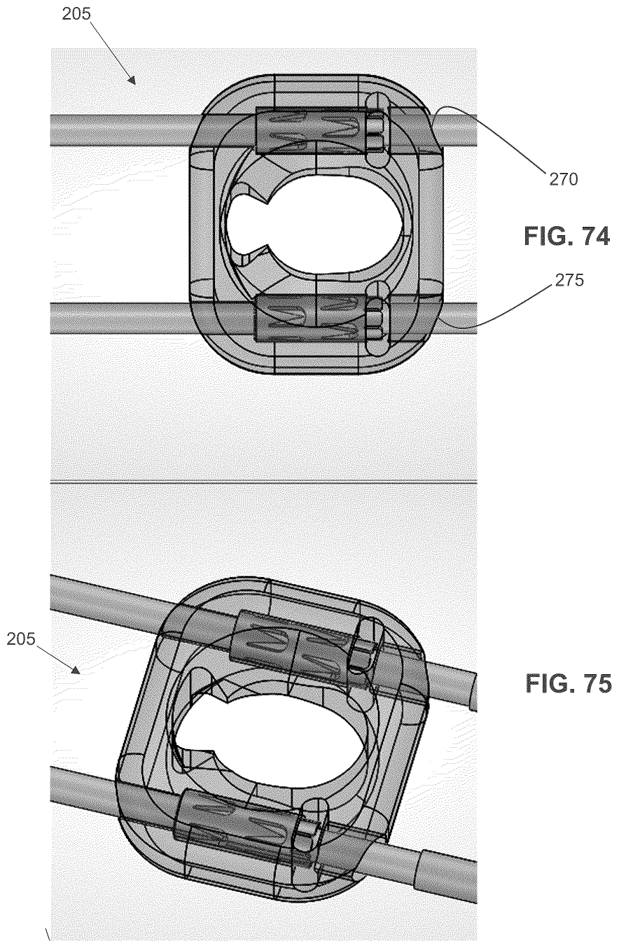

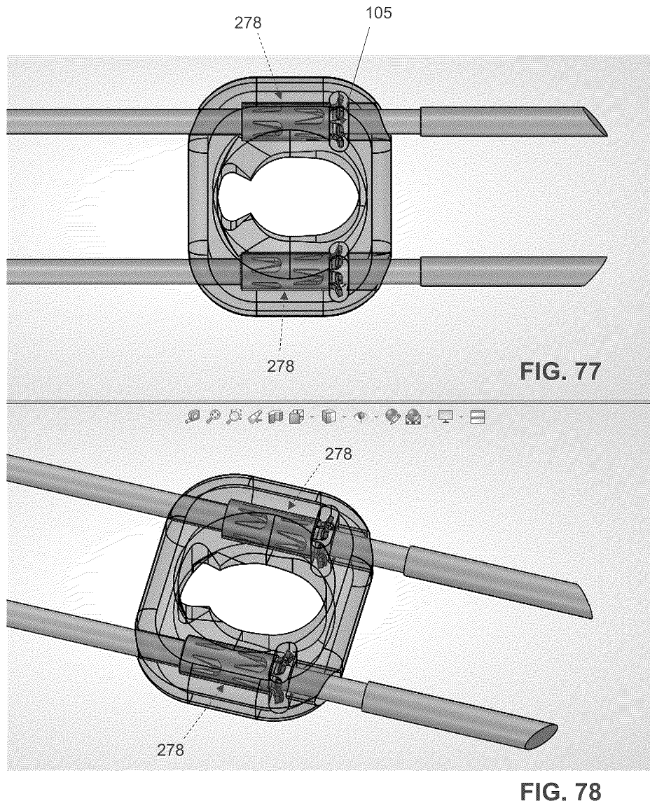

In another preferred form of the invention, and looking now at FIGS. 77 and 78, suture-securing mechanism 278 may also comprise protrusions 105 that may be positioned to engage an outer surface of bore 270 (or bore 275) of suture-locking washer 205 so as to maintain the position of suture-securing mechanism 278 relative to body 210 of suture-locking washer 205. In a preferred form of the invention, protrusions 105 are configured for insertion into bores 270, 275 in a first configuration (as shown in FIGS. 74 and 75) and then bent into a second configuration (as shown in FIGS. 77 and 78) so that protrusions 105 engage with portions of body 210 of suture-locking washer 205 adjacent to bore 270 (or bore 275) and/or engage with portions of bore 270 (or bore 275) themselves so as to secure suture-securing mechanisms 278 in bores 270, 275. Preferably, protrusions 105, first top window 280 and second top window 285 are configured so that protrusions 105 flare out from bores 270, 275 and extend into first top window 280 and second top window 285, whereby to lock suture-securing mechanisms 278 in bores 270, 275.

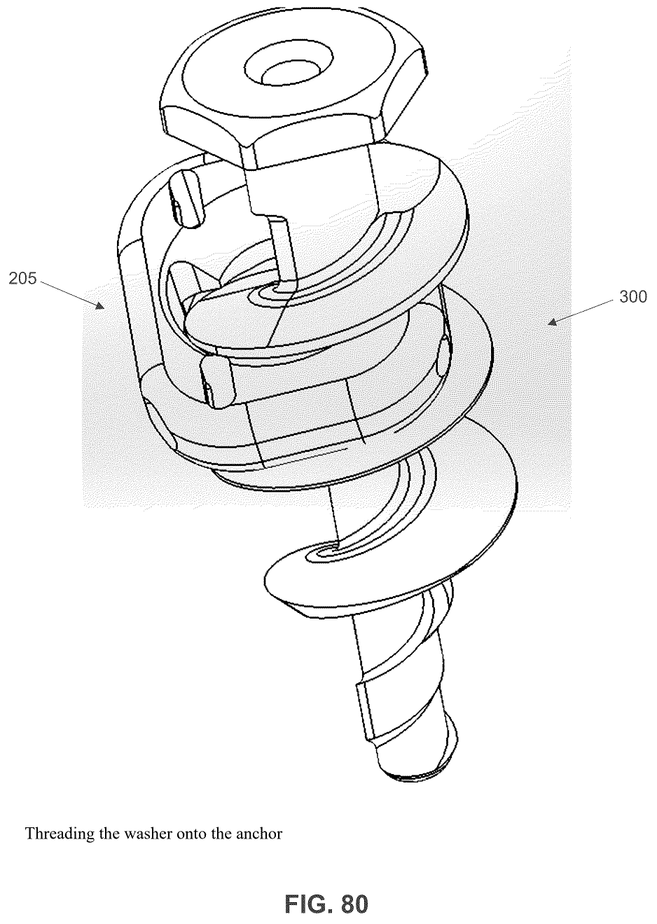

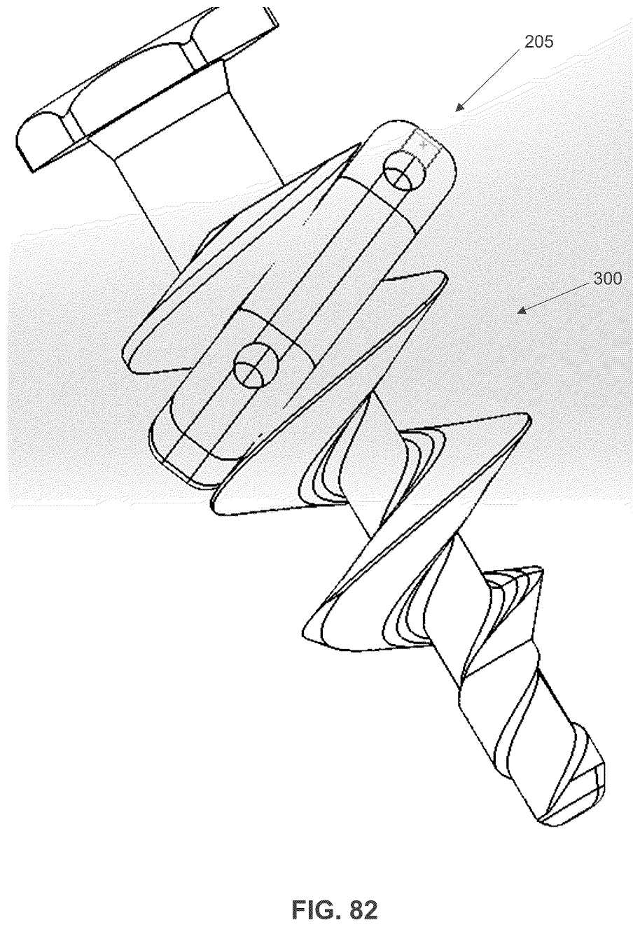

Looking now at FIGS. 79-84, central bore 245 of suture-locking washer 205 is configured to receive an anchor 248 therein, whereby to secure suture-locking washer 205 to anchor 248. More particularly, anchor 248 generally comprises a distal end 290, a proximal end 295, and a “corkscrew” body 300 extending therebetween. Body 300 is defined by a thread 305 having a generally “corkscrew-like” geometry, with thread 305 extending from distal end 290 to a head 310 formed on proximal end 295.

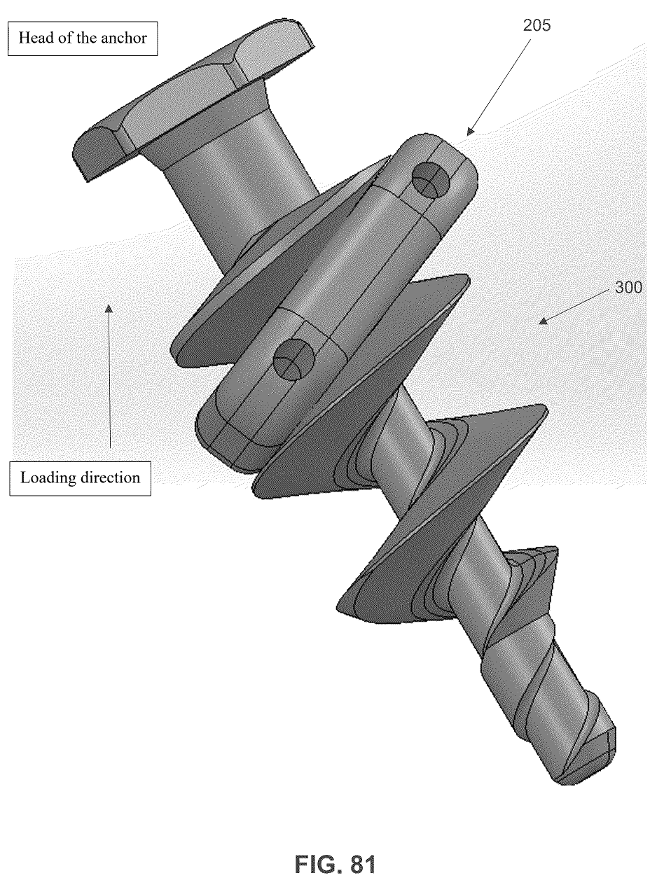

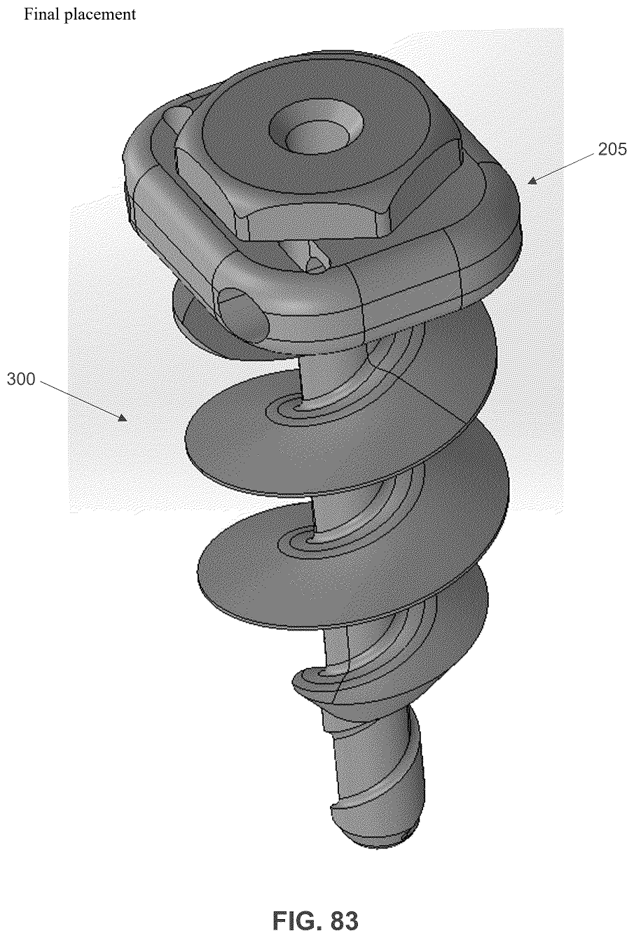

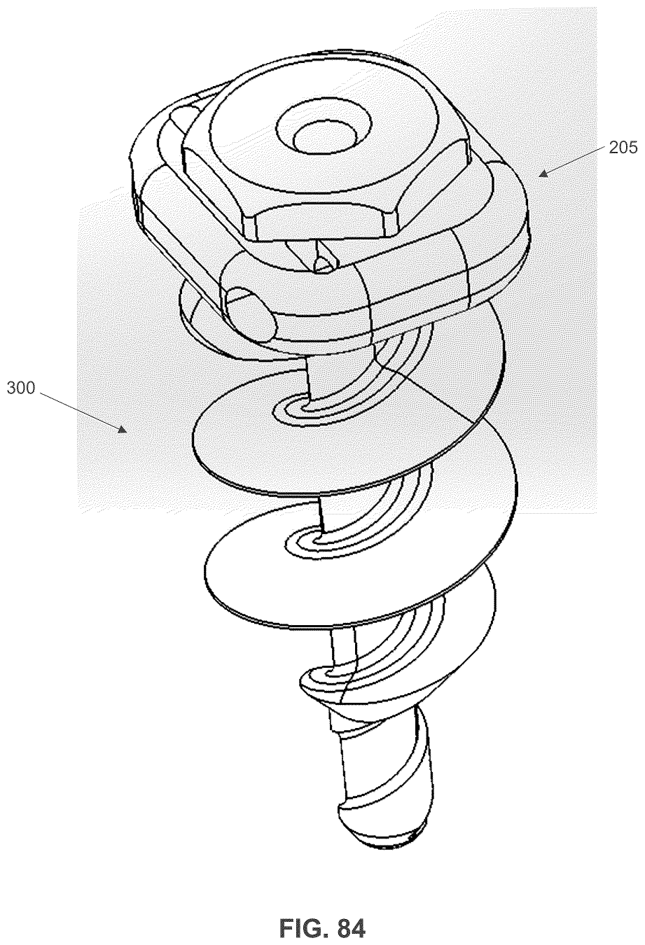

Anchor 248 may be mounted to suture-locking washer 205 (or vice versa) by inserting distal end 290 of anchor 248 into central bore 245 of suture-locking washer 205 and rotating anchor 248 (e.g., clockwise from the perspective of FIG. 79). As anchor 248 is rotated relative to suture-locking washer 205, thread 305 of anchor body 310 is received in side cutout 255 and directed downward by lead-in chamfered surface 265. Thus, as anchor 248 is rotated relative to suture-locking washer 205, thread 310 is passed under upper surface 215 of suture-locking washer 205 so as to contact lower surface 220 of suture-locking washer 205. When anchor 248 has been fully advanced distally relative to suture-locking washer 205, head 310 of anchor 248 is in contact with, and pushed against, upper surface 215 of suture-locking washer 205. See FIGS. 83 and 84. At this point, suture-locking washer 205 is mounted to anchor 248. Significantly, as can be seen in FIG. 81, the geometry of central bore 245 permits suture-locking washer 205 to tilt relative to anchor 248 as suture-locking washer 205 is initially mounted to, and/or rotates relative to, anchor 248, but to thereafter be fully seated and flush against head 310 of anchor 248 after anchor 248 has been fully advanced distally through central bore 245 (as can be seen FIGS. 83 and 84). The geometry of central bore 245 is specifically configured so as to provide a bore which is large enough to enable anchor 248 to be passed through suture-locking washer 205, but small enough for suture-locking washer 205 to still be held tightly to anchor 248 after assembly (i.e., if central bore 245 were to be configured as a slot, it could easily receive anchor 248, however, anchor 248 would be loosely disposed within central bore 245 after assembly).

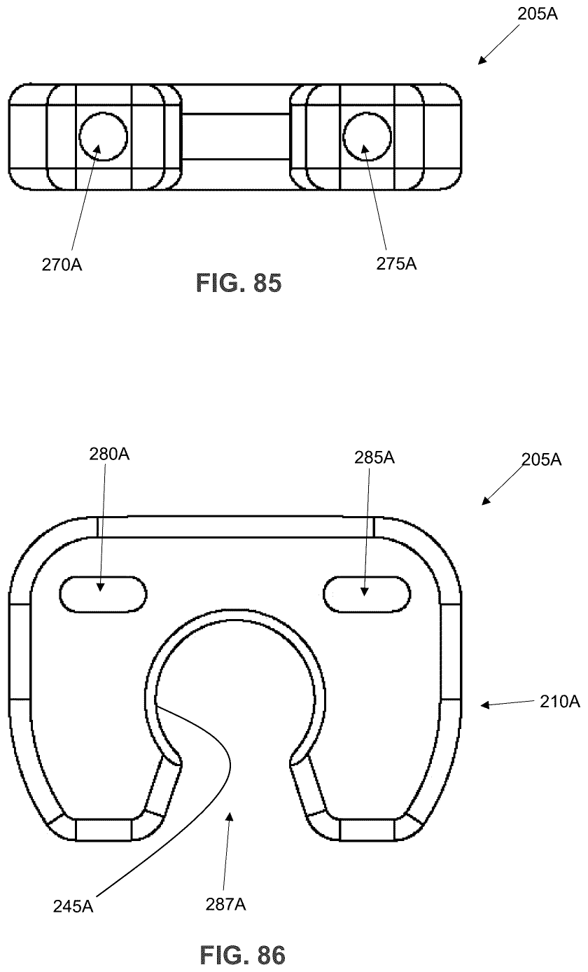

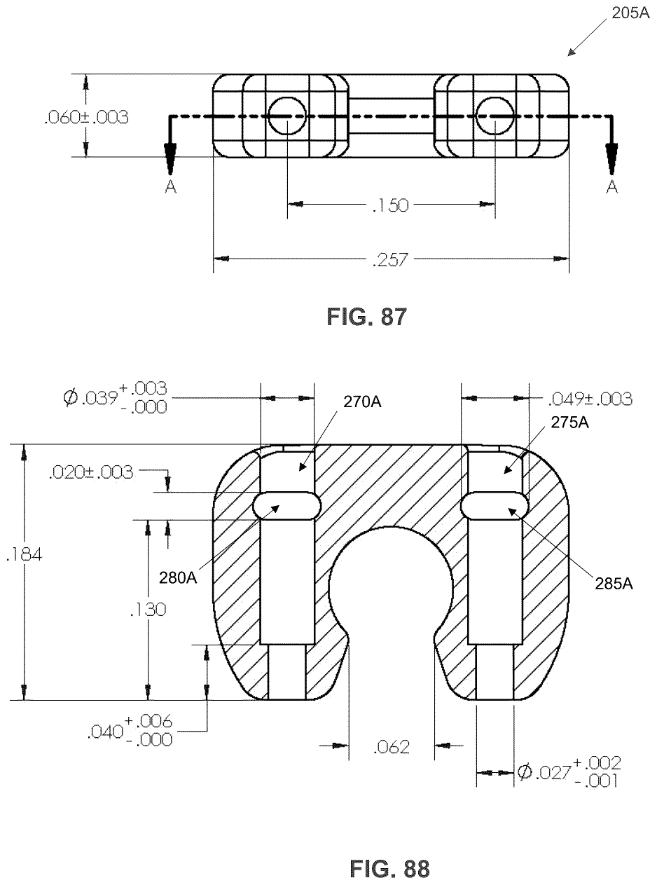

Looking next at FIGS. 85-92, there is shown another novel suture-locking washer 205A formed in accordance with the present invention. Suture-locking washer 205A is generally similar to the aforementioned suture-locking washer 205, however, the body 210A of suture-locking washer 205A is formed as a “U-shape” such that the central bore 245A of suture-locking washer 205A comprises an open lead-in gap 287A, such that suture-locking washer 205A may be attached to an anchor (e.g., anchor 248) by passing a portion of the anchor (e.g., body 300 of anchor 248) through gap 287A and into central bore 245A, as will be apparent to one of skill in the art in view of the present disclosure. Suture-locking washer 205A comprises bores 270A, 275A for receiving a suture-securing mechanism 278 (and/or suture-securing mechanisms 95, 95A, etc.). Suture-locking washer 205A also comprises first top window 280A and second top window 285A opening into bore 270A and bore 275A, respectively.

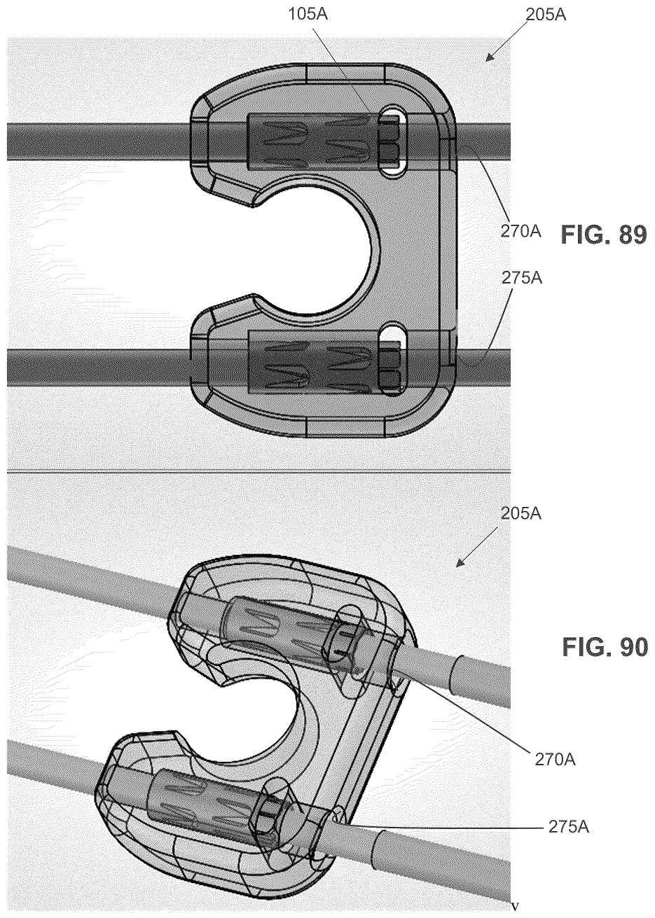

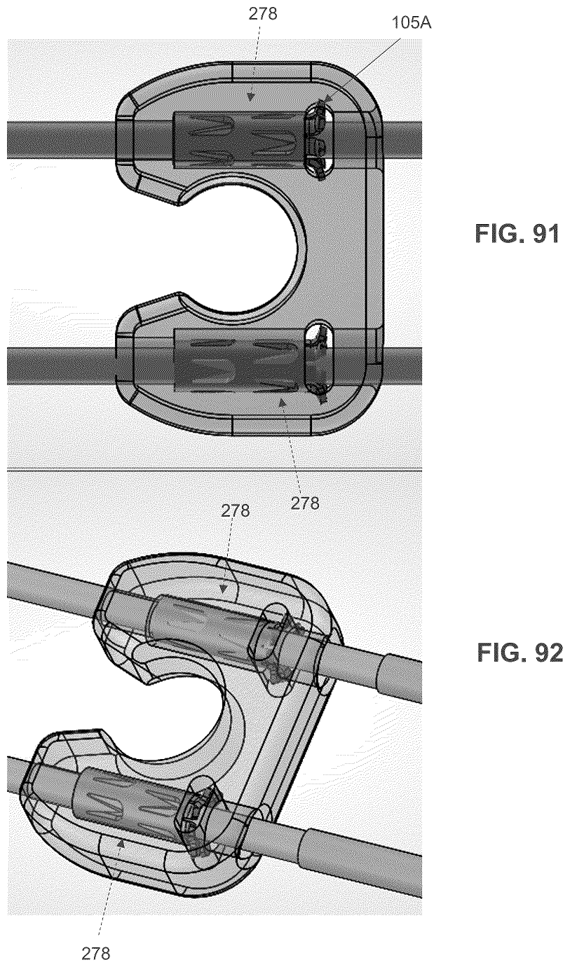

In a preferred form of the invention, and looking now at FIGS. 89-92, suture-securing mechanism 278 may also comprise protrusions 105 that may be positioned to engage an outer surface of bore 270A (or bore 275A) of suture-locking washer 205A so as to maintain the position of suture-securing mechanism 278 relative to body 210 of suture-locking washer 205A. In a preferred form of the invention, protrusions 105 are configured for insertion into bores 270A, 275A in a first configuration (as shown in FIGS. 89 and 90) and then bent into a second configuration (as shown in FIGS. 91 and 92) so that protrusions 105 engage with portions of body 210A of suture-locking washer 205A adjacent to bore 270A (or bore 275A) and/or engage with portions of bore 270A (or bore 275A) themselves so as to secure suture-securing mechanisms 278 in bores 270A, 275A. Preferably, protrusions 105, first top window 280A and second top window 285A are configured so that protrusions 105 flare out from bores 270A, 275A and extend into first top window 280A and second top window 285A, whereby to lock suture-securing mechanisms 278 in bores 270A, 275A.

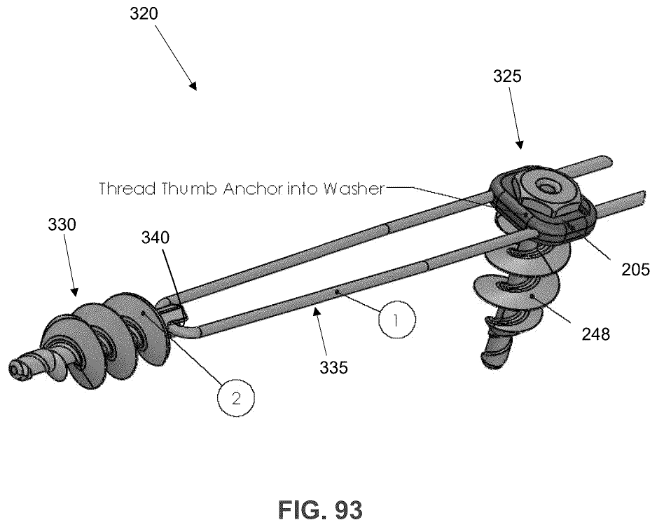

The present invention further comprises the provision and use of a suture securing system incorporating suture-locking washer 205 and two or more anchors. To that end, and looking now at FIG. 93, there is shown a novel suture securing system 320 formed in accordance with the present invention. Suture securing system 320 generally comprises a first anchoring element 325, a second anchoring element 330, and a suture 335 extending therebetween. First anchoring element 325 preferably comprises suture-locking washer 205 (or suture-locking washer 205A) mounted to a first anchor 248 in the manner discussed above, such that upper surface 215 of suture-locking washer 205 is disposed against head 310 of anchor 248. Second anchoring element 330 may comprise substantially any suitable anchor configured to slidably receive suture 335. By way of example but not limitation, second anchoring element 330 may comprise an anchor substantially similar to the aforementioned anchor 248 in which head 310 comprises a suture-receiving opening 340 for slidably receiving suture 335, as will hereinafter be discussed in further detail.

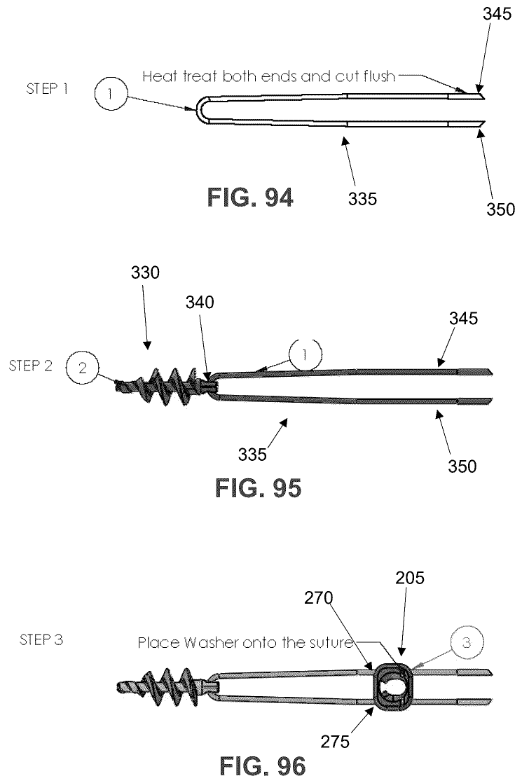

In one preferred method of use, and looking now at FIG. 94, suture 335 is formed into a loop having two free ends 345, 350 (which may be heat treated in order to facilitate mounting to suture-locking washer 205, as will hereinafter be discussed).

Looking next at FIG. 95, suture 335 is passed through suture-receiving opening 340 of head 310 so as to slidably mount second anchoring element 330 to suture 335.

Looking next at FIG. 96, suture-locking washer 205 is preferably mounted to suture 335 as follows. First, free end 345 of suture 335 is passed into bore 270 of suture-locking washer 205 so as to extend completely through bore 270 (i.e., so as to extend from front surface 225 of suture-locking washer 205 to rear surface 230 of suture-locking washer 205). Concurrently or subsequently, free end 335 of suture 335 is passed into bore 275 of suture-locking washer 205 so as to extend completely through bore 270 (i.e., so as to extend from front surface 225 of suture-locking washer 205 to rear surface 230 of suture-locking washer 205). As a result of this construction, suture-locking washer 205 is able to slide relative to suture 335.

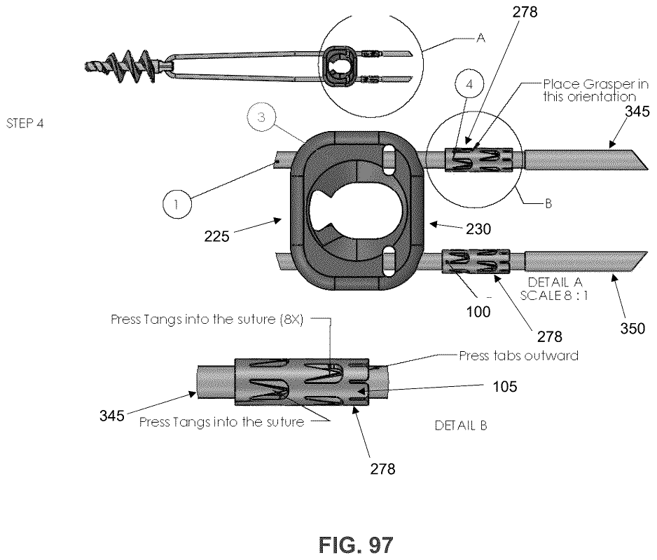

Looking next at FIG. 97, a first suture-securing mechanism 278 is mounted to suture 335 at a location between free end 345 of suture 335 and rear surface 230 of suture-locking washer 205. A second suture-securing mechanism 278 is mounted to suture 335 at a location between free end 350 of suture 335 and rear surface 230 of suture-locking washer 205. It will be appreciated that, inasmuch as suture securing mechanisms 278 are configured to permit movement of the suture to which they are mounted in only one direction, the suture securing mechanisms 278 are mounted such that free ends 345, 350 of suture 335 may be selectively tensioned so as to move suture 335 relative to suture securing mechanisms 278, however, movement of suture 335 in the opposite direction is arrested by suture-holding elements 100 (e.g., barbs or teeth) on suture securing mechanisms 278. Suture securing mechanisms 278 are also configured to not pass entirely through bores 270, 275, whereby to be received within bores 270, 275 without passing through bores 270, 275, as will hereinafter be discussed.

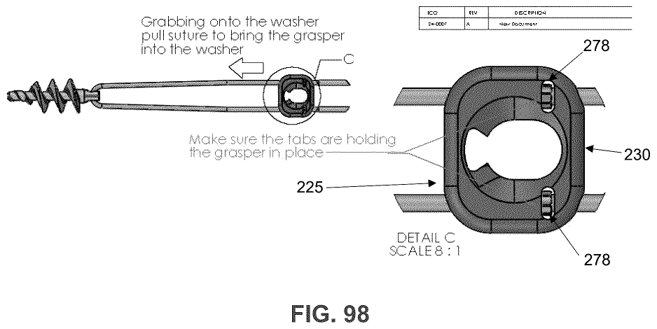

Looking next at FIG. 98, suture-locking washer 205 is held and free ends 345, 350 of suture 335 are pulled distally (i.e., towards second anchoring element 330) until suture securing mechanisms 278 are pulled into bores 270, 275 of suture-locking washer 205. Protrusions 105 on suture securing mechanisms 278 can then be bent outward to hold suture securing mechanisms 278 in place within bores 270, 275 of suture-locking washer 205. As a result of this construction, suture securing mechanisms 278 are retained within bores 270, 275 of suture-locking washer 205 and free ends 345, 350 of suture 335 may be pulled through suture securing mechanisms 278 (i.e., free ends 345, 350 may be pulled proximally (i.e., away from rear surface 230 of suture-locking washer 205) to shorten the length of suture 335 extending between second anchoring element 330 and suture-locking washer 205 of first anchoring element 325.

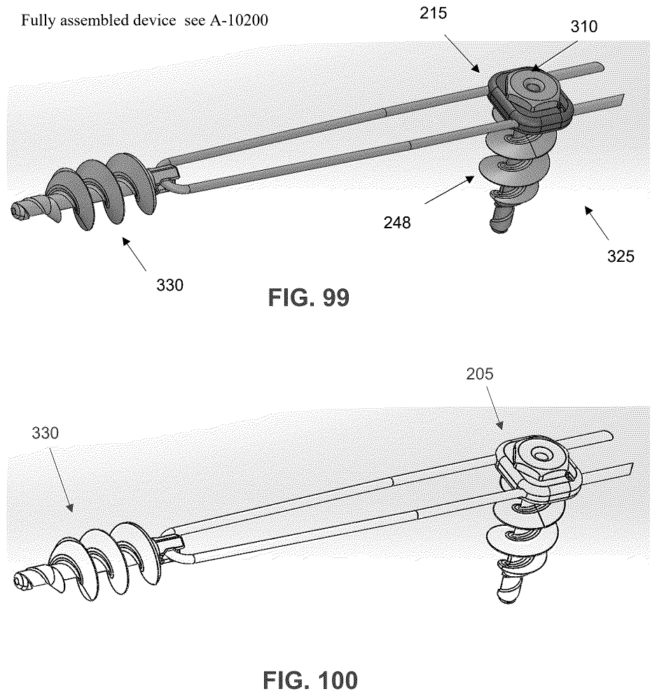

Looking next at FIGS. 99 and 100, anchor 248 of first anchoring element 325 is passed through central bore 245 (i.e., in the manner discussed above) such that upper surface 215 of suture-locking washer 205 is disposed against head 310 of anchor 248. At this point, first anchoring element 325 and second anchoring element 330 may be secured to a structure (e.g., soft tissue, bone, etc.) and free ends 345, 350 may be pulled to shorten the distance between first anchoring element 325 and second anchoring element 330, as will be apparent to one of skill in the art in view of the present disclosure.

Note that the exemplary procedure discussed above may be modified as desired without departing from the scope of the present invention. By way of example but not limitation, if desired, anchor 248 may be mounted to suture-locking washer 205 before suture 335 is passed through bores 270, 275 (and/or suture securing mechanisms 278) in the manner discussed above. And it should also be appreciated that the anchor of second anchoring element 330 and/or first anchoring element 325 may be mounted to a structure (e.g., soft tissue, bone, etc.) before suture 335 is passed through suture-receiving opening 340 of second anchoring element 330 and/or bores 270, 275 of suture-locking washer 205, as will be apparent to one of skill in the art in view of the present disclosure.

It has also been recognized that suture 335 may fail after suture securing system 320 is secured in the anatomy. By way of example but not limitation, where suture securing system 320 is used as a “sling” to tether a first bone to a second bone, failure of suture 335 (e.g., due to friction fraying the suture, etc.) causes the “sling” to fail.

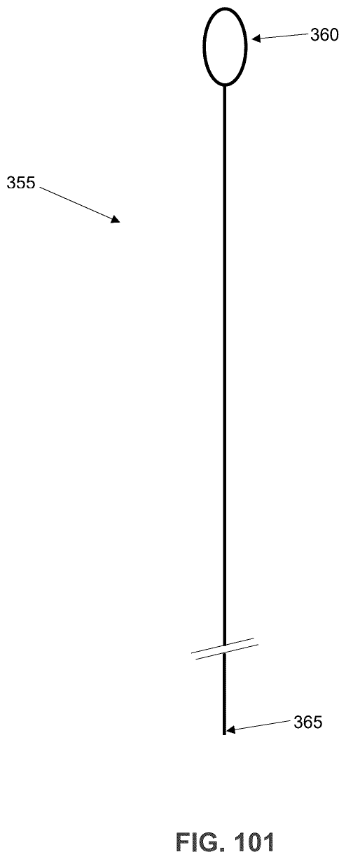

In order to address the foregoing possibility of failure of suture 335, and looking now at FIG. 101, in another embodiment of the invention, there is provided an augment suture 355. Augment suture 355 is formed as a suture having a loop 360 pre-formed on a first end of suture 355, and a free end 365 opposite loop 360. In a preferred form of the invention, loop 360 comprises a “noose” that closes down when free end 365 is pulled, as will be apparent to one of skill in the art in view of the present disclosure. Augment suture 355 is preferably used to connect first anchoring element 325 to soft tissue in the anatomy in which suture securing system 320 is deployed. By way of example but not limitation, augment suture 355 may be used to connect first anchoring element 325 to the joint capsule, the abductor pollicis longus (APL) muscle, the extensor pollicis brevis (EPB) muscle, etc., when suture securing system 320 is deployed in the hand of a patient. In a preferred form of the invention, augment suture 355 comprises a #0 suture.

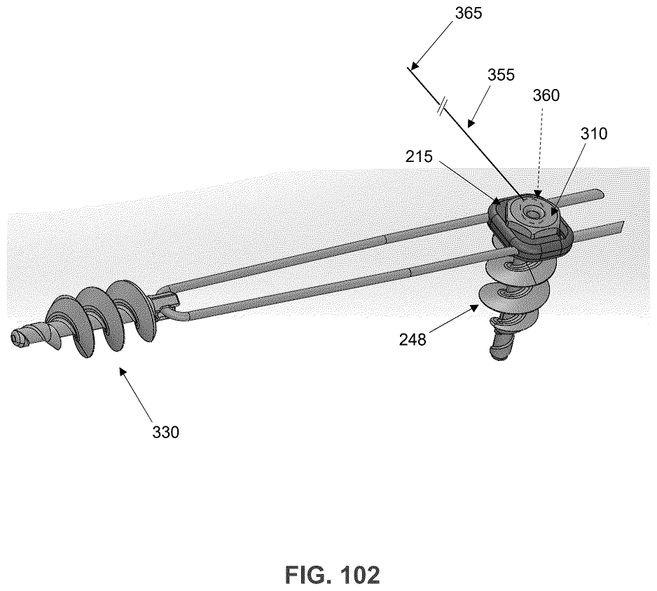

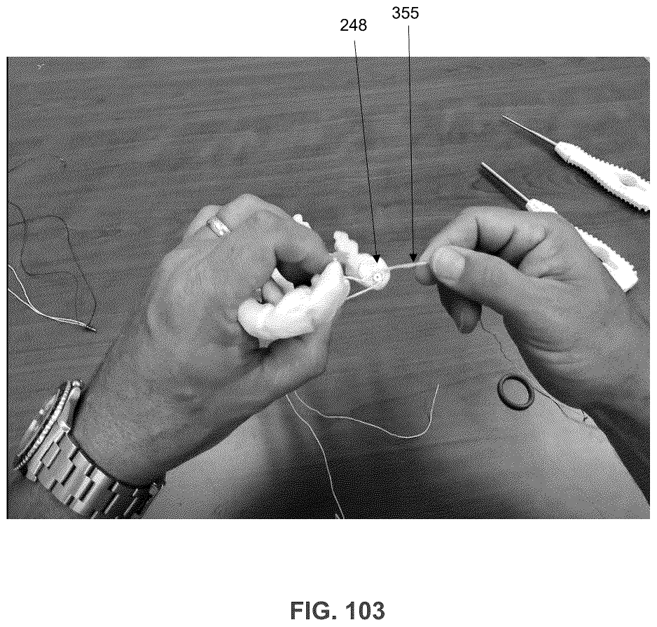

Looking now at FIGS. 102 and 103, augment suture 355 is preferably mounted to first anchoring element 325 by securing augment suture 355 between head 310 of anchor 248 and upper surface 215 of suture-locking washer 205.

More particularly, in accordance with one method of mounting augment suture 355 to first anchoring element 325, loop 360 is passed over head 310 of anchor 248. The surgeon then pulls free end 365 of augment suture 355 so as to close down loop 360 around anchor 248 and then mounts anchor 248 to suture-locking washer 205 in the manner discussed above. Next, anchor 248 and suture-locking washer 205 (with augment suture disposed between head 310 of anchor 248 and upper surface 215 of suture-locking washer 205) are then inserted into the anatomy in the manner discussed above. Free end 365 of augment suture 355 can then be secured to the soft tissue in the anatomy in a manner that will be apparent to one of skill in the art in view of the present disclosure.

It will be appreciated that, as a result of the foregoing construction, augment suture 355 is disposed between head 310 and upper surface 215 of suture-locking washer 205, protecting loop 360 from friction with surrounding anatomy that may cause fraying. Furthermore, using the (preferably flat) upper surface of head 310 of anchor 248 to secure augment suture 355 eliminates the need to use a projection or eyelet on head 310 (e.g., a suture-securing head 340) projecting into the anatomy.

### Bone Anchor Comprising Suture-Securing Mechanisms

If desired, suture-securing mechanisms 95, 95B may be incorporated directly into a bone anchor, e.g., holes 103 may be formed in the head of a bone anchor, and suture-securing mechanisms 95, 95B may be disposed in holes 103. In this way, a bone anchor may be provided with a suture-locking facility without requiring the use of a separate element (i.e., a suture-locking washer).

### Other Medical Devices Comprising Suture-Securing Mechanisms

If desired, suture-securing mechanisms 95,95B may be incorporated into other medical devices, e.g., bone plates, etc. In this form of the invention, holes 103 may be formed in the medical device, and suture-securing mechanisms 95, 95B may be disposed in holes 103. In this way, a medical device may be provided with a suture-locking facility.

### Applications

In addition to the foregoing, it should also be appreciated that the present invention may be used in basal joint arthroplasties which may be conducted for purposes other than alleviating basal joint arthritis, e.g., such as in the case of disease and/or trauma.

Also, suture-locking washer 75 (and suture-locking washers 75A, 75B, 75C, 75D and 205) and suture-securing mechanisms 95 (and suture-securing mechanism 95B) may be applicable to alternative applications that use bone anchors or other applications which use implantable medical devices. By way of example but not limitation, suture-locking washers 75 (and suture-locking washers 75A, 75B, 75C, 75D and 205) may be utilized to provide knotless suture attachment to screw anchors during virtually all orthopedic procedures requiring fixation of suture, utilized to reposition or support the position of tendon, or other soft tissue, relative to bone.

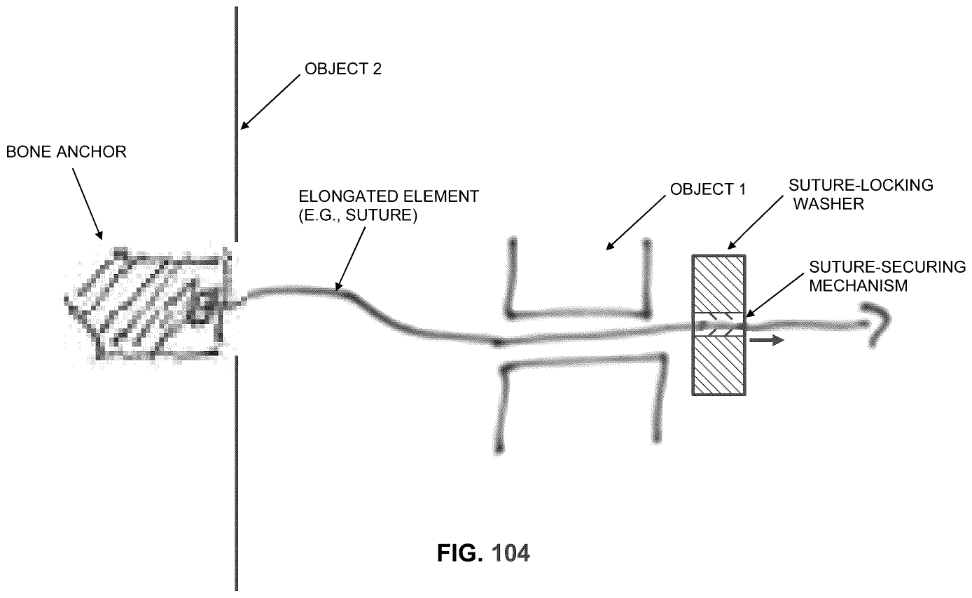

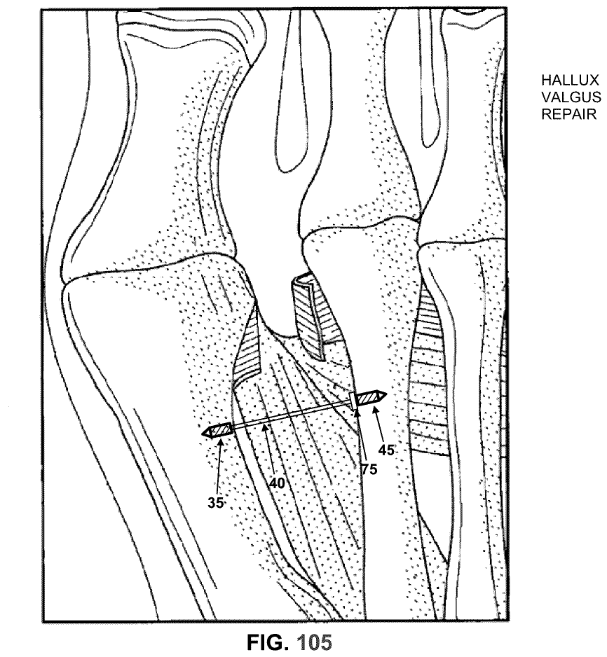

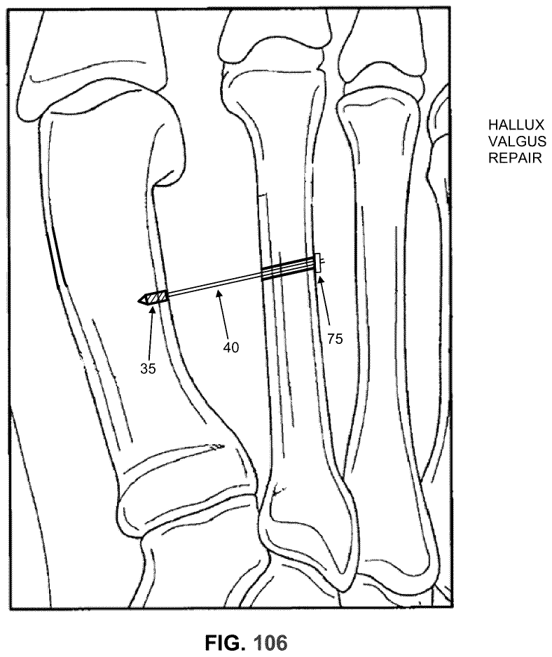

More particularly, and looking now at FIG. 104, where it is desired to secure a first object (e.g., a bone, soft tissue, etc.) to a second object (e.g., a bone, etc.), a bone anchor is disposed in the second object, the suture extending away from the bone anchor is passed through an opening in the first object, and then the suture-locking washer 75 (or suture locking washer 75A, 75B, 75C 75D or 205), containing a suture-securing mechanism 95 (or suture-securing mechanism 95B), is loaded onto the free end of the suture and pushed against the far side of the first object so as to cinch the first object against the second object. In this way, the bone anchor, suture and suture-locking washer can be used to secure the first object to the second object.

Applications of the apparatus and methods of the present invention include, but are not limited to rotator cuff repair, tenodesis, medial (ulnar) collateral ligament reconstruction (the “Tommy John Procedure”), lateral ulnar collateral ligament reconstruction, Carpal instability treated with scapholunate and lunotriquetral ligament reconstructions (Blatt Capsulodesis), thumb carpometacarpal arthroplasty (ligament reconstruction with tendon interposition—LRTI), chronic thumb ulnar collateral ligament reconstruction (Gamekeeper's thumb), chronic thumb radial collateral ligament reconstruction, finger metacarpophalangeal ligament reconstruction, medial collateral ligament repair/reconstructions with autograft or allograft, lateral collateral ligament repair/reconstruction with autograft or allograft, posterolateral reconstruction with autograft or allograft, various lateral collateral ligament reconstructions (Watson-Jones/Chrisman Snook), or other applications involving securing suture strand(s) to bone in order to secure tendons or other soft tissue to bone.

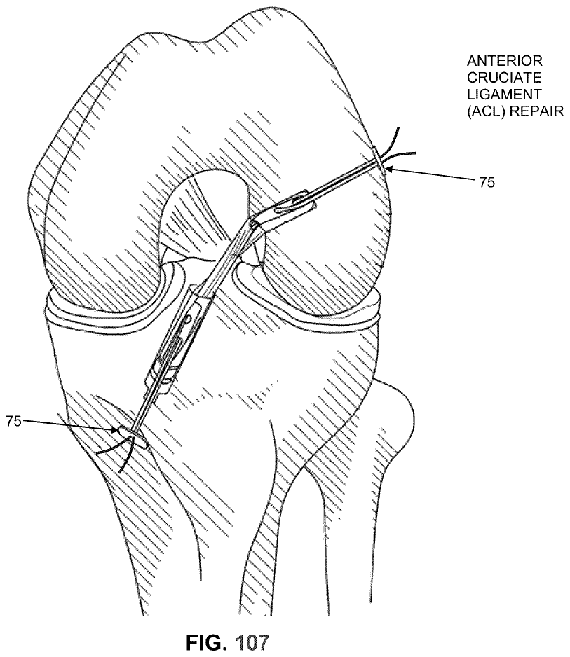

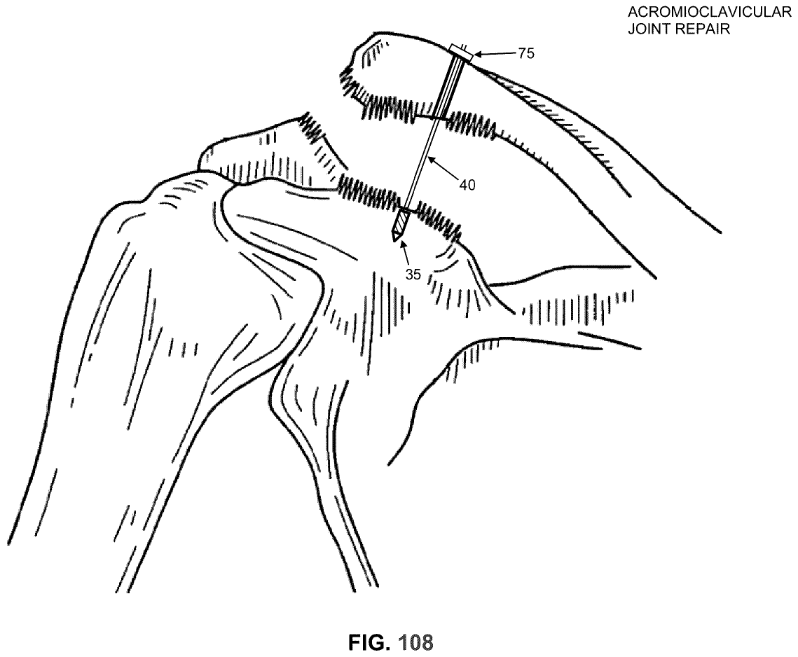

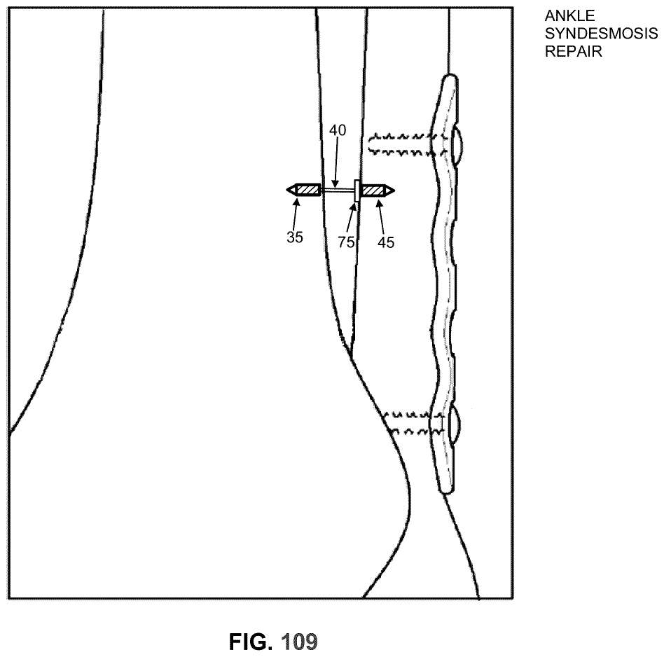

By way of example but not limitation, see FIG. 105 which shows a hallux valgus repair using the apparatus and methods of the present invention; FIG. 106 which shows another hallux valgus repair using the apparatus and methods of the present invention; FIG. 107 which shows an anterior cruciate ligament (ACL) repair using the apparatus and methods of the present invention; FIG. 108 which shows acromioclavicular joint repair using the apparatus and methods of the present invention; and FIG. 109 which shows an ankle syndesmosis repair using the apparatus and methods of the present invention.

### Nomenclature

In the foregoing disclosure, there is disclosed a sling suspension system for supporting the thumb of a patient after basal joint arthroplasty, and the sling suspension system is disclosed as comprising a sling which may comprise a suture. In addition, in the foregoing disclosure, there are disclosed apparatus and methods for effecting other orthopedic repairs, wherein the apparatus comprises a sling or suture. In view of this, in order to render the text more readable, various components have been referred to herein in the context of suture, e.g., washers 75A, 75B, 75C, 75D and 205 are sometimes referred to as “suture-locking” washers, mechanisms 95 and 95B are sometimes referred to as “suture-securing” mechanisms, and elements 100 are sometimes referred to as “suture-holding” elements.

However, it should be appreciated that the apparatus and methods of the present invention are applicable to a wide range of applications where elongated elements other than suture per se need to be secured in place. By way of example but not limitation, such other elongated elements might comprise non-suture filaments. It will be appreciated by those skilled in the art that washers 75A, 75B, 75C, 75D and 205, mechanisms 95 and 95B, and elements 100 are equally capable of locking or securing or holding non-suture elongated elements and filaments. Therefore, it will be appreciated that suture-locking washers 75A, 75B, 75C, 75D and 205 may also be appropriately referred to as “elongated element-locking” washers or “filament-locking” washers, suture-securing mechanisms 95 and 95B may also be appropriately referred to as “elongated element-securing” mechanisms or “filament-securing” mechanisms, and suture-holding elements 100 may also be appropriately referred to as “elongated element-holding” elements or “filament-holding” elements.

Therefore, where the context so admits, suture-locking washers 75A, 75B, 75C, 75D and 205 should also be considered to be “elongated element-locking” washers or “filament-locking” washers, suture-securing mechanisms 95 and 95B should also be considered to be “elongated element-securing” mechanisms or “filament-securing” mechanisms, and suture-holding elements 100 should also be considered to be “elongated element-holding” elements or “filament-holding” elements.

### Modifications

It is to be appreciated that the present invention is by no means limited to the particular constructions herein disclosed and/or shown in the drawings, but also comprises any modifications or equivalents within the scope of the invention. Although the present invention has been described in terms of the preferred embodiments above, numerous modifications and/or additions to the above-described preferred embodiments would be readily apparent to one skilled in the art.

What is claimed is:

1. Apparatus for adjustably securing an object to a washer body, the apparatus comprising:
a washer body having a central opening;
a first passageway extending through the washer body on a first side of the central opening and a second passageway extending through the washer body on a second, opposite side of the central opening; and
a first suture-securing structure disposed in the first passageway and a second suture-securing structure disposed in the second passageway, wherein each of the first suture-securing structure and the second suture-securing structure comprise a side wall defining a suture passageway, a portion of the side wall projecting into the passageway so as to provide one-way movement of a suture through the suture passageway.

2. The apparatus according to claim 1 further comprising a first window extending into the first passageway and a second window extending into the second passageway.

3. The apparatus according to claim 2 wherein the first and second suture-securing structures are configured to extend out of the first and second passageways in the washer body to secure the first and second suture-securing structures in the first and second passageways in the washer body.

4. The apparatus according to claim 3 wherein the first tubular structure comprises at least one end projection configured to be bent into engagement with the first window to secure the first suture-securing structure in the first passageway in the washer body, and further wherein the second suture-securing structure comprises at least one end projection configured to be bent into engagement with the second window to secure the second suture-securing structure in the second passageway in the washer body.

5. The apparatus according to claim 1 wherein the central opening comprises a side cutout.

6. The apparatus according to claim 1 wherein a portion of the washer body surrounding the central opening is formed with decreasing thickness along an axis parallel to a central longitudinal axis of the central opening so as to form a lead-in chamfered surface.

7. The apparatus according to claim 2 wherein the central opening comprises a longitudinal axis and the first passageway and the second passageway extend perpendicular to the longitudinal axis of the central opening.

8. The apparatus according to claim 7 wherein the first window and the second window extend parallel to the longitudinal axis of the central opening.

9. The apparatus according to claim 1 wherein the washer body is generally rectangular in shape.

10. The apparatus according to claim 1 wherein the washer body comprises a bone anchor.

11. The apparatus according to claim 1 wherein the apparatus further comprises a suture extending through the first suture passageway and the second suture passageway.

12. The apparatus according to claim 1 wherein the apparatus further comprises a bone anchor extending through the central opening of the washer body.

13. A method for securing a first anatomical object to a second anatomical object, the method comprising:
providing an apparatus for securing the first anatomical object to the second anatomical object, the apparatus comprising a first anchor body, a second anchor body and a suture connecting the first anchor body to the second anchor body, wherein a length of the suture extending between the first anchor and the second anchor is adjustable;
positioning the first anchor body in the first anatomical structure so as to secure the first anchor body to the first anatomical structure;
positioning the second anchor body in the second anatomical structure so as to secure the second anchor body to the second anatomical structure; and
adjusting the suture so as to shorten the length of suture extending between the first anatomical object and the second anatomical object so as to fixedly connect the first anatomical object to the second anatomical object.

14. The method according to claim 13 wherein the first anchor body is positioned against a far surface of the first anatomical object.

15. The method according to claim 14 wherein the suture extends to the far surface of the first anatomical object by passing the suture through a through-hole formed in the first anatomical object.

16. The method according to claim 13 wherein the second anchor body is positioned against a far surface of the second anatomical object.

17. The method according to claim 16 wherein the suture extends to the far surface of the second anatomical object by passing the suture through a through-hole formed in the second anatomical object.

18. The method according to claim 13 wherein the first anatomical object comprises the index metacarpal of a patient and the second anatomical object comprises the thumb metacarpal of the patient.

19. The method according to claim 13 wherein at least one of the first anchor body and the second anchor body comprises:
a washer body having a central opening;
a first passageway extending through the washer body on a first side of the central opening and a second passageway extending through the washer body on a second, opposite side of the central opening; and
a first suture-securing structure disposed in the first passageway and a second suture-securing structure disposed in the second passageway, wherein each of the first suture-securing structure and the second suture-securing structure comprise a side wall defining a suture passageway, a portion of the side wall projecting into the passageway so as to provide one-way movement of the suture through the suture passageway.

20. The method according to claim 19 further comprising passing a first end of the suture through the first suture-securing structure and passing a second end of the suture through the second suture-securing structure, passing the first suture-securing structure through the first passageway and passing the second suture-securing structure through the second passageway.

21. The method according to claim 20 further comprising bending a portion of the first suture-securing structure to secure the first suture-securing structure in the first passageway and bending a portion of the second suture-securing structure to secure the second suture-securing structure in the second passageway.

22. The method according to claim 19 wherein the washer body is mounted to one of the first anatomical structure and the second anatomical structure before the suture is inserted into the first and second passageways of the washer body.

23. The method according to claim 19 wherein the washer body is mounted to one of the first anatomical structure and the second anatomical structure after the suture is inserted into the first and second passageways of the washer body.

24. The method according to claim 19 further comprising passing a bone anchor through the central opening of the washer body to mount the washer body to one of the first anatomical structure and the second anatomical structure.
